(19) United States
(12) Patent Application Publication
(10) Pub. No.: US 2017/0136089 A1
(43) Pub. Date: May 18, 2017

(54) COMPOSITIONS AND METHODS OF REGULATING BONE RESORPTION

(71) Applicant: THE TRUSTEES OF THE UNIVERSITY OF PENNSYLVANIA, Philadelphia, PA (US)

(72) Inventors: George HAJISHENGALLIS, Philadelphia, PA (US); Toshiharu ABE, Nagoya, Aichi (JP); Jieun SHIN, Philadelphia, PA (US)

(73) Assignee: THE TRUSTEES OF THE UNIVERSITY OF PENNSYLVANIA, Philadelphia, PA (US)

(21) Appl. No.: 15/311,103

(22) PCT Filed: May 12, 2015
(86) PCT No.: PCT/US2015/030339

§ 371 Date: Nov. 14, 2016

Related U.S. Application Data
(60) Provisional application No. 61/993,339, filed on May 15, 2014.

(51) Int. Cl. A61K 38/18; A61K 38/19; A61K 9/00; C07K 14/485; A61K 45/06; A61K 38/20

(52) U.S. Cl. CPC A61K 38/1808; A61K 45/06; A61K 38/204; A61K 9/0019; C07K 14/485; A61K 38/191; A61K 38/20; A61K 9/0063; C07K 2319/30

(57) ABSTRACT

The present invention includes compositions and methods that take advantage of MFG-E8's role as a homeostatic regulator of osteoclasts for treating a bone disorder or regulating osteoclast activation. In one aspect, the invention includes a composition comprising an effective amount of milk fat globule-EGF factor 8 (MFG-E8) formulated for local administration and a pharmaceutically acceptable carrier or adjuvant. In another aspect, a method is included for regulating osteoclast activation and inhibiting bone loss at a target site comprising locally administering an effective amount of milk fat globule-EGF factor 8 (MFG-E8) to the target site.

### CROSS-REFERENCE TO RELATED APPLICATION

The present application is entitled to priority under 35 U.S.C. §119(e) to U.S. Provisional Patent Application No. 61/993,339, filed May 15, 2014, which is hereby incorporated by reference in its entirety herein.

### STATEMENT REGARDING FEDERALLY SPONSORED RESEARCH OR DEVELOPMENT

This invention was made with government support under DE015254, DE017138, and DE021685, awarded by the National Institutes of Health. The government has certain rights in the invention.

### BACKGROUND OF THE INVENTION

Osteoclasts are large multinuclear cells which function to resorb bone tissue. Bone resorption is a normal process which occurs in coordination with bone formation, a process in which osteoblasts are involved. Essentially, osteoclasts break down bone and release minerals into the blood while osteoblasts form new bone tissue.

Bone diseases, such as osteoporosis, metastatic bone disease, rheumatoid arthritis, peridontal bone disease and Paget's disease, are characterized by a loss of bone. In many cases, bone loss leads to weak bones that are susceptible to fracturing. In addition to the pain and suffering, patients can become physically impaired which often leads to complications having negative consequences on patient health and quality of life. Moreover, the economic costs attributable to these diseases are tremendous.

Receptors and ligands of the Tumor Necrosis Factor family have recently been shown to play an essential part in the differentiation and activity of osteoclasts. Tumor Necrosis Factor-α (TNF-α) is known to promote osteoclastogenesis. The utilization of TNF-α antagonists, such as monoclonal antibodies, for therapeutic purposes, has proven difficult, however, because of immunity to the large molecule, and limited entry into some specialized compartments of the body.

There is a need for preventing bone loss and treating bone disorders by regulating osteoclastogenesis and the resorbing activity of mature osteoclasts.

### SUMMARY OF THE INVENTION

As described herein, the present invention includes compositions and methods that take advantage of MFG-E8's role as a homeostatic regulator of osteoclasts for treating a bone disorder or regulating osteoclast activation. One aspect of the invention includes a composition comprising an effective amount of milk fat globule-EGF factor 8 (MFG-E8) formulated for local administration and a pharmaceutically acceptable carrier or adjuvant.

Another aspect of the invention includes a composition for treating a bone disorder comprising an effective amount of milk fat globule-EGF factor 8 (MFG-E8) formulated for local administration.

In another aspect, the invention includes a method of regulating osteoclast activation at a target site comprising locally administering an effective amount of milk fat globule-EGF factor 8 (MFG-E8) to the target site.

In yet another aspect, the invention includes a method of inhibiting bone loss at a target site comprising locally administering an effective amount of milk fat globule-EGF factor 8 (MFG-E8) to the target site.

In still another aspect, the invention includes a method of inhibiting inflammation at a target site comprising locally administering an effective amount of milk fat globule-EGF factor 8 (MFG-E8) to the target site.

In another aspect, the invention includes a method of treating a bone disorder comprising locally administering an effective amount of milk fat globule-EGF factor 8 (MFG-E8) to a target site.

In still another aspect, the invention includes use of a composition comprising an effective amount of milk fat globule-EGF factor 8 (MFG-E8) formulated for local administration and a pharmaceutically acceptable carrier or adjuvant for treating a bone disorder.

In various embodiments of the above aspects or any other aspect of the invention delineated herein, the composition is formulated for oral administration, such as a liquid suspension, a chewable composition, and an orally disintegrating tablet or capsule composition. In one embodiment, the composition is formulated for delayed-release. In another embodiment, the composition is formulated to target a site of bone loss. In yet another embodiment, the target site is selected from the group consisting of periodontal tissue, alveolar process, arthritic and non-arthritic joints, an injured bone, and other bone sites. In another embodiment, the target site is the periodontal tissue and the administration is in a gingival tissue.

In another embodiment, the effective amount comprises in a range of about 0.1 μg/ml to about 2 μg/ml per single dose. In one embodiment, the effective amount inhibits at least one condition selected from the group consisting of osteoclastogenesis, inflammation and bone resorption.

In another embodiment, the composition further comprises at least one binder, excipient, diluent, or any combination thereof. In yet another embodiment, the composition further comprises at least one of an antimicrobial agent and an anti-inflammatory agent.

In one embodiment, the administration inhibits expression of at least one osteoclast marker, such as NFATc1, cathepsin K, and αvβ3 integrin. In one embodiment, the administration inhibits osteoclastogenesis. In another embodiment, the administration inhibits RANKL-induced osteoclastogenesis. In yet another embodiment, the administration inhibits bone resorption. In still another embodiment, the administration inhibits expression of at least one bone resorption stimulator, such as a bone resorption stimulator comprising TNF, IL-6, IL-17A, MMP-9, Ptgs2, RANKL, IL-17A, Tnfsf11 1, CXCL1, CXCL2, CXCL3, CXCL5, and combinations thereof. In another embodiment, the administration inhibits expression of at least one proinflammatory cytokine selected from the group consisting of IL-8 and CCL2/MCP-1.

One embodiment of the invention includes inhibition that decreases expression of at least one inflammation molecule selected from the group consisting of a pro-inflammatory mediator, an adhesion molecule and an immune receptor. In this embodiment, the at least one molecule is selected from the consisting of IL-6, IL-8, IL-17a, MMP9, PTGS2, TNFSF11, SPP1, CSF3, CXCL1, CXCL2, CXCL3, CXCL5, ITGAL, SELE, CXCR2, CCR1, and TREM1. In another embodiment, the inhibition suppresses periodontal microbiota growth.

In another embodiment, the bone disorder is selected from the group consisting of osteoporosis, osteomalacia, osteosclerosis, and osteopetrosis.

### BRIEF DESCRIPTION OF THE DRAWINGS

The following detailed description of preferred embodiments of the invention will be better understood when read in conjunction with the appended drawings. For the purpose of illustrating the invention, there are shown in the drawings exemplified embodiments. It should be understood, however, that the invention is not limited to the precise arrangements and instrumentalities of the embodiments shown in the drawings.

FIG. 1A is a graph showing a timecourse of MFG-E8 mRNA expression in the periodontal tissue after ligature-induced periodontitis, determined by real-time PCR. Results (means±SD; n=5 mice) were normalized to GAPDH mRNA and presented relative to those at dO, set as 1. *P<0.01. NS, not significant.

FIG. 1B is a graph showing TRAP+MNCs enumeration and averages (with SD) from 60 random coronal sections (20 from each of three mice per group) of ligated teeth with surrounding periodontal tissue from the mice of FIG. 1A. The dashed line with open circles shows the timecourse of bone loss in similarly treated mice (same mice used in A; means±SD for n=5).

FIG. 1C is a panel of images of tissue sections from ligature-induced periodontitis at d5 were stained as indicated. Bottom row stainings involve the same section processed for immunofluorescence followed by TRAP staining. B, bone; D, dentin; DIC, differential interference contrast; PL, periodontal ligament. Scale bars; 50 μm (white), 500 μm (black).

FIG. 1D is a panel of graphs showing mRNA expression of indicated molecules by real-time PCR of RANKL-induced OCLs from progenitor RAW264.7 cells. Results (means±SD; n=3) were normalized to GAPDH mRNA and presented relative to those of undifferentiated RAW264.7 cells, set as 1. *P<0.01.

FIG. 1E is a panel of images showing light microscopy of undifferentiated and RANKL-differentiated RAW264.7 cells (scale bar, 50 μm) and a graph showing the enumeration of TRAP+MNCs in the cultures (means±SD; n=3). *P<0.01.

FIG. 1F is a graph showing anti-MFG-E8 immunoblotting of cell lysates from undifferentiated RAW264.7 cells (−RANKL) and differentiated OCLs (+RANKL).

FIG. 1G is a panel of images showing DIC and fluorescent images of RANKL-differentiated OCL stained for MFG-E8 and nuclei (DAPI) (scale bar, 50 μm).

FIG. 1H is a blot showing immunoprecipitation of MFG-E8 from culture supernatants of RANKL-differentiated OCLs using goat anti-MFG-E8 IgG antibody (1, OCLs differentiated from RAW264.7 cells; 2, OCLs differentiated from mouse BM-derived precursors) followed by immunoblotting with the same antibody.

FIG. 2 is a panel of graphs showing that MFG-E8 inhibits the expression of osteoclast differentiation and activation markers. RANKL-induced osteoclastogenesis from progenitor RAW264.7 cells was performed in the absence or presence of the indicated concentrations of MFG-E8 and mRNA expression of the indicated molecules was assayed by real-time PCR. Results (means±SD; n=4) were normalized to GAPDH mRNA and presented relative to those of undifferentiated RAW264.7 cells, set as 1. *P<0.05; **P<0.01.

FIG. 3A is a graph showing the enumeration of TRAP+MNCs in RANKL-stimulated cultures of WT and Mfge8−/− OCPs and representative photomicrographs after TRAP staining (scale bar, 100 μm).

FIG. 3B is a graph showing mRNA expression of the indicated molecules by real-time PCR from RANKL-induced OCLs generated from WT or Mfge8−/−OCPs.

Results were normalized to those of GAPDH mRNA and presented relative to those of undifferentiated OCPs, set as 1. *P<0.05 compared with control.

FIG. 3C is a graph showing total resorbed area in each culture as measured and expressed relative to the WT group, set as 1 and representative images of WT and Mfge8−/−OCPs cultured under osteoclastogenic conditions for 4 d on Ca3(PO4)2-coated wells and resorptive areas (dark spots) as visualized by light microscopy.

FIG. 3D is a panel of a graph of RANKL-induced osteoclastogenesis from Mfge8−/−OCPs in the presence of increasing MFG-E8 concentrations by counting TRAP+MNCs and representative images of cells stained for TRAP expression. The dashed line marks the number of OCLs formed from WT OCPs (no exogenous MFG-E8 added). Data are means±SD (A,B,D, n=3; C, n=6). **P<0.01 compared with control.

FIG. 4A is a panel of graphs showing mRNA expression of the indicated molecules assayed by qPCR. Results were normalized to GAPDH mRNA and presented relative to those of undifferentiated monocytes (not treated with RANKL), assigned an average value of 1. n=3. *P<0.05, **P<0.01 compared to untreated control.

FIG. 4B is a panel of images showing human MFG-E8 inhibits RANKL-induced osteoclastogenesis from human monocytes and resorption pit formation. Human CD14+ monocytes underwent RANKL-induced osteoclastogenesis for 5 d in the presence of the indicated increasing concentrations of MFG-E8 (−RANKL indicates control monocytes not subjected to osteoclastogenesis). Cells were stained for TRAP expression and TRAP+ multinucleated cells (MNCs) were counted (right). Data are means±SD (n=3). *P<0.01.

FIG. 4C is a panel of images showing human CD14+ monocytes on Ca3(PO4)2-coated wells (Osteo Assay Surface plates; Corning) cultured under osteoclastogenic conditions (as above) and resorptive areas (dark spots) visualized by light microscopy and a graph showing the total resorbed area in each culture measured and expressed relative to the WT group, set as 1. Data are means±SD (n=8). *P<0.01.

FIG. 5A is a graph showing periodontal bone loss was induced for 5 d in WT or Mfge8−/− mice by ligating a maxillary second molar and leaving the contralateral tooth unligated (baseline control).

FIG. 5B is a panel of graphs showing WT and Mfge8−/− mice treated as described herein and TRAP+MNCs enumerated from 20 random coronal sections of the ligated second molar from each of three mice (upper left graph) and averaged with SD from the total 60 sections per group (upper right graph) and images of sections stained with TRAP, hematoxylin, orange-G, and aniline blue indicate OCLs adjacent to bone (right). Scale bars; 100 μm (white), 500 μm (black).

FIG. 5C is a graph showing mRNA expression from real-time PCR of indicated molecules from dissected gingiva from mice used in FIG. 3A. Results were normalized to GAPDH mRNA and presented as fold change in the transcript levels in ligated sites relative to those of unligated sites (assigned an average value of 1).

FIG. 5D is a graph showing mRNA expression from real-time PCR of indicated molecules from dissected gingiva from mice used in FIG. 3A. Results were normalized to GAPDH mRNA and presented as fold change in the transcript levels in ligated sites relative to those of unligated sites (assigned an average value of 1).

FIG. 5E is a graph showing periodontal bone loss in mice locally microinjected with 5 μg anti-MFG-E8 mAb or IgG2a control Id before placing the ligature and every day thereafter until the day before sacrifice (d5).

FIG. 5F is a graph showing naturally-occurring bone loss in 13-month-old Mfge8−/− mice and age-matched WT controls relative to bone measurements in 10-wk-old WT mice (0 baseline).

FIG. 5G is a graph showing periodontal bone loss in mice locally microinjected with 2.5 μg MFG-E8 or BSA control as outlined in D.

FIG. 5H is a graph showing mRNA expression of the indicated molecules as outlined in FIG. 3C from dissected gingiva from mice used in FIG. 3G. Data are means±SD (n=5-8 mice per group, except for B; n=3). *P<0.05; **P<0.01 compared with control or between indicated groups.

FIG. 5I is a graph showing mRNA expression of the indicated molecules as outlined in FIG. 3C from dissected gingiva from mice used in FIG. 3G. Data are means±SD (n=5-8 mice per group, except for B; n=3). *P<0.05; **P<0.01 compared with control or between indicated groups.

FIG. 6A is a graph showing the measurement of mineral density of mouse tibiae. Tissue mineral density of tibiae from wild-type (WT) or MFG-E8-deficient (Mfge8−/−) mice was measured by means of micro-computed tomography. Data are means±SD (n=3 mice per group). HA, hydroxyapatite. *P<0.05.

FIG. 6B is a panel of reconstructed images the scanned regions of WT and Mfge8−/− tibiae.

FIG. 7 is a panel of images showing time-dependent increase of osteoclast numbers upon ligature-induced periodontitis in mice. Groups of mice were subjected to ligature-induced periodontitis and were euthanized at the indicated days. Maxillae with intact surrounding tissue were processed for histological staining. Arrows in coronal sections stained with TRAP, hematoxylin, orange-G, and aniline blue indicate osteoclasts (TRAP+ multinucleated cells) adjacent to bone. A magnification that would allow easy visualization of osteoclasts was used (scale bar; 100 μm). Osteoclasts were also enumerated and averaged (with SD) from a total of 60 random coronal sections per group (results shown in FIG. 1B).

FIG. 8A is a panel of blots showing NFATc1 expression in RANKL-differentiated osteoclasts. Anti-NFATc1 immunoblottings of cell lysates from undifferentiated osteoclast precursors (−RANKL) and RANKL-differentiated osteoclasts (+RANKL). Left image, RAW264.7 cells; right image, osteoclast precursors from bone marrow of wild-type (WT) or MFG-E8-deficient (Mfge8−/−) mice.

FIG. 8B is a panel of blots showing MFG-E8expression in RANKL-differentiated osteoclasts. Anti-MFG-E8 immunoblottings of cell lysates from undifferentiated osteoclast precursors (−RANKL) and RANKL-differentiated osteoclasts (+RANKL). Left image, mouse bone marrow-derived osteoclast precursors; right image; human osteoclast precursors from CD14+ monocytes.

FIG. 9 is a graph showing MFG-E8 inhibits bone loss in Mfge8−/− mice following ligature-induced periodontitis. Periodontal bone loss was induced for 5 d in groups of Mfge8−/− mice by ligating a maxillary second molar and leaving the contralateral tooth unligated (baseline control). The mice were locally microinjected with 2.5 μg MFG-E8 or BSA Id before placing the ligature and every day thereafter until the day before sacrifice (d5). Data are means±SD (n=5-6 mice per group). *P<0.01.

FIG. 10A is a graph showing that MFG-E8 reduces the periodontal bacterial burden without exerting direct antimicrobial activity. WT or Mfge8−/− mice were subjected to ligature-induced periodontitis for 5 d until the age of 13 months as described herein. Bacteria were extracted from recovered ligatures and serial dilutions of bacterial suspensions were plated onto blood agar plates for anaerobic growth and CFU enumeration. Each symbol represents an individual mouse and small horizontal lines indicate the mean. *P<0.01.

FIG. 10B is a graph showing that MFG-E8 reduces the periodontal bacterial burden without exerting direct antimicrobial activity. WT or Mfge8−/− mice were subjected to naturally-occurring periodontitis until the age of 13 months for 5 d until the age of 13 months as described herein. Oral swabs held against the gumlines were taken and bacteria were cultured anaerobically for CFU enumeration. Each symbol represents an individual mouse and small horizontal lines indicate the mean. *P<0.01.

FIG. 10C is a graph showing ligature-induced periodontitis in WT mice performed with or without local treatment with 2.5 μg MFG-E8, as described herein. Bacteria were extracted from recovered ligatures and serial dilutions of bacterial suspensions were plated onto blood agar plates for anaerobic growth and CFU enumeration. Each symbol represents an individual mouse and small horizontal lines indicate the mean. *P<0.01.

FIG. 10D is an image showing possible antimicrobial activity of MFG-E8 against mouse periodontal bacteria as determined by the disk inhibition zone method, using PBS and imipenem as negative and positive control, respectively. The experiment shown is representative of a total of 15 bacterial isolates and MFG-E8 consistently failed to inhibit bacterial growth. Numbers shown refer to g of compound used.

FIG. 11A is a graph showing MFG-E8 expression in human periodontal ligament cells (HPDL) after TNF-α stimulation.

FIG. 11B is a blot showing MFG-E8 expression in HPDL cells after TNF-α stimulation.

FIG. 12A is a panel of graphs showing MFG-E8 (upper left graph) and DEL-1 (upper right graph) expression in stem maxilla cells and MFG-E8 (lower left graph) and DEL-1 (lower right graph) expression in HPDL cells treated with siRNA to MFG-E8 and/or Del-1.

FIG. 12B is a panel of blots showing MFG-E8 and DEL-1 expression in stem cell maxilla (upper blot) and HPDL (lower blot) cells treated with siRNA to MFG-E8 and/or DEL-1.

FIG. 13A is a panel of graphs showing secreted levels of IL-6 (left), IL-8 (middle) and CCL2/MCP-1 (right) in HPDL cells treated with siRNA to MFG-E8 and/or DEL-1, but lacked TNF-α stimulation.

FIG. 13B is a panel of graphs showing secreted levels of IL-6 (left), IL-8 (middle) and CCL2/MCP-1 (right) in HPDL cells treated with siRNA to MFG-E8 and/or DEL-1 and TNF-α stimulation.

FIG. 14A is a panel of graphs showing secreted levels of IL-6 (left), IL-8 (middle) and CCL2/MCP-1 (right) in stem maxilla cells treated with siRNA to MFG-E8 and/or DEL-1, but lacked TNF-α stimulation.

FIG. 14B is a panel of graphs showing secreted levels of IL-6 (left), IL-8 (middle) and CCL2/MCP-1 (right) in stem maxilla cells treated with siRNA to MFG-E8 and/or DEL-1 and TNF-α stimulation.

FIG. 15 is a graph showing recovery of IL-8 levels in HPDL cells with treatment of rhMFG-E8.

FIG. 16A is a graph showing MFG-E8-Fc decreases inflammatory clinical parameters, probing pocket depth, of non-human primate (NHP) periodontitis.

FIG. 16B is a graph showing MFG-E8-Fc decreases inflammatory clinical parameters, gingival index, of non-human primate (NHP) periodontitis.

FIG. 16C is a graph showing MFG-E8-Fc decreases inflammatory clinical parameters, bleeding on probing, of non-human primate (NHP) periodontitis.

FIG. 16D is a graph showing MFG-E8-Fc decreases inflammatory clinical parameters, mobility index, of non-human primate (NHP) periodontitis.

FIG. 17 is a panel of graphs showing inhibition of periodontal bone loss after treatment of non-human primate periodontitis with MFG-E8-Fc.

FIG. 18 is a flowchart showing distribution of subjects and sites in each group. GCF testing was done at baseline for all groups. Repeated testing was done after non-surgical treatment and surgical treatment for a subgroup (30 sites) from the Chronic Severe Periodontitis group.

FIG. 19A is a graph showing the level of MFG-E8 among the participant study groups.

FIG. 19B is a graph showing the level of IL-17A among the participant study groups.

FIG. 19C is a graph showing the level of IL-6 among the participant study groups.

FIG. 19D is a graph showing the level of RANKL among the participant study groups.

FIG. 19E is a graph showing the level of OPG among the participant study groups.

FIG. 19F is a graph showing the level of IL-1β among the participant study groups.

FIG. 19G is a graph showing the level of IL-1α among the participant study groups.

FIG. 19H is a graph showing the level of PPD among the participant study groups.

FIG. 20 is a scatter plot showing correlation between PPD and MFG-E8 in all subjects at initial examination. Significant negative correlation found between PPD and MFG-E8 (r-coefficient=−0.7101, p-value<0.0001).

FIG. 21A is a graph showing change in probing pocket depths (PPD) in the treated severe periodontitis subgroup at initial examination, re-assessment and post-operation.

FIG. 21B is a graph showing MFG-E8 levels in the treated severe periodontitis subgroup at initial examination, re-assessment and post-operation.

FIG. 21C is a graph showing IL-17A levels in the treated severe periodontitis subgroup at initial examination, re-assessment and post-operation.

FIG. 21D is a graph showing IL-6 levels in the treated severe periodontitis subgroup at initial examination, re-assessment and post-operation.

FIG. 21E is a graph showing RANKL levels in the treated severe periodontitis subgroup at initial examination, re-assessment and post-operation.

FIG. 22 is a graph showing total MFG-E8 levels among the participant study groups.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or within 10%, 9%, 8%, 7%, 6%, 5%, 4%, 3%, 2%, 1%, 0.5%, 0.1%, 0.05%, or 0.01% of the specified value, as such variations are appropriate to perform the disclosed methods. Unless otherwise clear from context, all numerical values provided herein are modified by the term about.

By “bone disorder” is meant a pathological disorder, disease, or condition in a mammal in which there is an imbalance in the ratio of bone formation to bone resorption, such that, if left untreated, would result in that mammal exhibiting an abnormal mass of bone. In an exemplary embodiment, the bone disorder is associated with decreased bone mass, osteoclastic resorption outweighing osteoblastic bone formation resulting in bone loss. Examples of bone disorders include, but are not limited to, osteoporosis, osteomalacia, osteosclerosis, and osteopetrosis.

By “bone regeneration” is meant a physiological process of bone formation, which can be due to fracture or injury healing, or the continuous remodelling that occurs throughout adult life of the bone.

The term “bone-related disorder” refers to any type of bone disease, the treatment of which may benefit from the administration of osteogenic lineage or bone-forming cells, e.g., osteoprogenitors, osteoblasts or osteocytes, to a subject having the disorder. In particular, such disorders may be characterised, e.g., by decreased bone formation or excessive bone resorption, by decreased number, viability or function of osteoblasts or osteocytes present in the bone, decreased bone mass in a subject, thinning of bone, compromised bone strength or elasticity, etc

By “bone resorption” is meant the process by which osteoclasts break down bone and release minerals, resulting in the transfer of calcium from the bone to the blood.

By “inflammation” is meant a biological response, such as increased expression and activation of pro-inflammatory mediators, adhesion molecules and immune receptors, to harmful foreign substances.

By “inflammation molecule” is meant any protein or polynucleotide having an alteration in expression level or activity that is associated with inflammation. Examples of inflammation molecules include, but are not limited to, pro-inflammatory mediators, adhesion molecules and immune receptors, such as IL-6, IL-17a, MMP9, PTGS2, TNFSF11, SPP1, CSF3, CXCL1, CXCL2, CXCL3, CXCL5, ITGAL, SELE, CXCR2, CCR1, and TREM1.

By “local administration” is meant local delivery of MFG-E8 by injection or application of MFG-E8 to the target site. In some embodiments, local administration refers to administration of MFG-E8 directly to a specific organ or tissue (e.g. periodontal tissue). Local administration may be achieved via injection of MFG-E8 directly into a bone or in the vicinity of the bone. Local administration may be achieved by topical administration of MFG-E8 at or near the target site. Local administration may be achieved by implantation of a device to deliver MFG-E8 at or near a target site by stereotactic surgery. Local administration may be achieved by implantation of MFG-E8 at or near an injured bone at a target site.

By “milk fat globule-EGF factor 8” or “MFG-E8” or “lactadherin” is meant a protein that is involved in a wide variety of cellular interactions, removal of apoptic cells, epithelial homeostasis, mucosal healing, neovascularization and cell adhesion. An exemplary MFG-E8 sequence includes human MFG-E8 found at GenBank Accession No. NM_001114614 and NP_001108086 (Q08431), or a fragment thereof, and the mouse MFG-E8 sequence found at NM_001045489 or NP_001038954, or a fragment thereof.

By “molecule” is meant any pro-inflammatory mediator, adhesion molecule or immune receptor. Examples include, but are not limited to, IL-6, IL-17a, MMP9, PTGS2, TNFSF11, SPP1, CSF3, CXCL1, CXCL2, CXCL3, CXCL5, ITGAL, SELE, CXCR2, CCR1, and TREM1, and combinations thereof.

By “osteoclast marker” is meant any protein or polynucleotide having an alteration in expression level or activity that is associated with osteoclast activation, osteoclast differentiation or osteoclastgenesis. Examples of osteoclast markers include, but are not limited to, NFATc1, cathepsin K, and αvβ3 integrin.

By “osteoclastogenesis” is meant the generation of new osteoclasts.

By “RANKL-induced osteoclastogenesis” is meant osteoclast formation that is induced by RANKL signaling and activation of downstream pathways required for osteogenesis.

By “target site” is meant a site within the body that is in need of treatment. Examples of such target sites includes, but is not limited to, periodontal tissue, alveolar process, arthritic and non-arthritic joints, an injured bone, bone in need of bone growth, bone in need of bone regeneration, and other bone sites.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “antimicrobial agent” is meant an agent that inhibits or stabilizes the proliferation or survival of a microbe. In one embodiment, a bacteriostatic agent is an antimicrobial. In other embodiments, any agent that kills a microbe (e.g., bacterium, fungus, virus) is an antimicrobial.

By “anti-inflammatory agent” is meant an agent that reduces the severity or symptoms of an inflammatory reaction in a tissue. An inflammatory reaction within tissue is generally characterized by leukocyte infiltration, edema, redness, pain, and/or fibrosis. Inflammation can also be measured by analyzing levels of inflammatory cytokines or any other pro-inflammatory markers.

By “binder, excipient, diluent” is meant a non-active ingredient. Non-active ingredients may solubilize, suspend, thicken, dilute, emulsify, stabilize, preserve, protect, color, flavor, and/or fashion active ingredients into an applicable and efficacious preparation, such that it may be safe, convenient, and/or otherwise acceptable for use. Examples of excipients include, but are not limited to, solvents, carriers, diluents, binders, fillers, sweeteners, aromas, pH modifiers, viscosity modifiers, antioxidants, extenders, humectants, disintegrating agents, solution-retarding agents, absorption accelerators, wetting agents, absorbents, lubricants, coloring agents, dispersing agents, and preservatives. Excipients may have more than one role or function, or may be classified in more than one group; classifications are descriptive only and are not intended to be limiting. In some embodiments, for example, the at least one excipient may be chosen from corn starch, lactose, glucose, microcrystalline cellulose, magnesium stearate, polyvinylpyrrolidone, citric acid, tartaric acid, water, ethanol, glycerol, sorbitol, polyethylene glycol, propylene glycol, cetylstearyl alcohol, carboxymethylcellulose, and fatty substances such as hard fat or suitable mixtures thereof.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence, or alteration in expression level or relative expression level of a transcriptional target to be detected.

A “dose” means the measured quantity of an active agent to be taken at one time by a patient.

A “dosage form” means a unit of administration of an active agent. Examples of dosage forms include tablets, capsules, injections, suspensions, liquids, emulsions, creams, ointments, suppositories, inhalable forms, transdermal forms, and the like.

By “effective amount” is meant the amount required to reduce or improve at least one symptom relative to an untreated patient. The effective amount of active compound(s) used to practice the present invention for therapeutic treatment of a disease varies depending upon the manner of administration, the age, body weight, and general health of the subject.

“Efficacy” means the ability of an active agent administered to a patient to produce a therapeutic effect in the patient.

The term “expression” as used herein is defined as the transcription and/or translation of a particular nucleotide sequence driven by its promoter.

By “fragment” is meant a portion of a polynucleotide or nucleic acid molecule. This portion contains, preferably, at least 10%, 20%, 30%, 40%, 50%, 60%, 70%, 80%, or 90% of the entire length of the reference nucleic acids. A fragment may contain 10, 20, 30, 40, 50, 60, 70, 80, 90, or 100, 200, 300, 400, 500, 600, 700, 800, 900, 1000, 1500, 2000 or 2500 (and any integer value in between) nucleotides. The fragment, as applied to a nucleic acid molecule, refers to a subsequence of a larger nucleic acid. A “fragment” of a nucleic acid molecule may be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

As used herein, the term “inhibit” or “inhibition” is meant to refer to a decrease in a biological state. For example, the term “inhibit” may be construed to refer to the ability to negatively regulate the expression, stability or activity of a protein, including but not limited to transcription of a protein mRNA, stability of a protein mRNA, translation of a protein mRNA, stability of a protein polypeptide, a protein post-translational modifications, a protein activity, a protein signaling pathway or any combination thereof.

“Instructional material,” as that term is used herein, includes a publication, a recording, a diagram, or any other medium of expression that may be used to communicate the usefulness of the compounds of the invention. In some instances, the instructional material may be part of a kit useful for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the compounds of the invention or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutical composition” or “pharmaceutically acceptable composition” refers to a mixture of at least one compound or molecule useful within the invention with a pharmaceutically acceptable carrier. The pharmaceutical composition facilitates administration of the compound or molecule to a patient. Multiple techniques of administering a compound or molecule exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound or molecule useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which may be hydrolyzed into the monomeric “nucleotides.” The monomeric nucleotides may be hydrolyzed into nucleosides. As used herein polynucleotides include, but are not limited to, all nucleic acid sequences that are obtained by any means available in the art, including, without limitation, recombinant means, i.e., the cloning of nucleic acid sequences from a recombinant library or a cell genome, using ordinary cloning technology and PCR™, and the like, and by synthetic means. The following abbreviations for the commonly occurring nucleic acid bases are used. “A” refers to adenosine, “C” refers to cytosine, “G” refers to guanosine, “T” refers to thymidine, and “U” refers to uridine. The term “RNA” as used herein is defined as ribonucleic acid. The term “recombinant DNA” as used herein is defined as DNA produced by joining pieces of DNA from different sources.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

As used herein, the terms “prevent,” “preventing,” “prevention,” and the like refer to reducing the probability of developing a disorder or condition in a subject, who does not have, but is at risk of or susceptible to developing a disorder or condition.

By “reduces” or “decreases” is meant a negative alteration of at least 10%, 25%, 50%, 75%, or 100%.

By “reference” is meant a standard or control. A “reference” is also a defined standard or control used as a basis for comparison.

As used herein, “sample” or “biological sample” refers to anything, which may contain the transcriptional target (e.g., polypeptide, polynucleotide, or fragment thereof) for which a transcriptional target assay is desired. The sample may be a biological sample, such as a biological fluid or a biological tissue. In one embodiment, a biological sample is a tissue sample including pulmonary vascular cells. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

As used herein, the term “sensitivity” is the expression level of the transcriptional target-detected in subjects with a particular disease.

A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, the terms “treat,” treating,” “treatment,” and the like refer to reducing or improving a disorder and/or symptom associated therewith. It will be appreciated that, although not precluded, treating a disorder or condition does not require that the disorder, condition or symptoms associated therewith be completely ameliorated or eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DESCRIPTION

The present invention includes compositions and methods that take advantage of the role of glycoprotein milk fat globule-EGF factor 8 (MFG-E8) plays as a homeostatic regulator of osteoclasts for treating a bone disorder or regulating osteoclast activation. The invention described herein includes compositions and methods for regulating osteoclast activation and inhibiting bone loss at a target site by administering MFG-E8.

The glycoprotein milk fat globule-EGF factor 8 (MFG-E8) is expressed in a range of tissues and mediates diverse functions including apoptotic cell clearance, angiogenesis, and repair of intestinal mucosa. Originally identified as a milk protein, milk fat globule-EGF-factor 8 (MFG-E8) is expressed in various tissues where it performs diverse homeostatic functions. Extensive research in mouse models of physiology and disease has shown that MFG-E8 mediates apoptotic cell clearance, maintenance and repair of intestinal epithelia, anti-inflammatory action in neutrophils and macrophages, and regulation of physiological (or pathological) angiogenesis. In both humans and animal models, the expression of MFG-E8 declines considerably in inflammatory conditions, including sepsis, colitis, acute lung injury, ischemia/reperfusion injury, atherosclerosis, and Alzheimer's disease.

MFG-E8 is expressed by and regulates osteoclasts, giant multinucleated cells (MNCs) that resorb bone during normal bone remodeling and also under pathologic inflammatory conditions (e.g., rheumatoid arthritis and periodontitis). The invention described herein capitalizes on the role played by MFG-E8 as a novel regulator to restrain RANKL-induced osteoclastogenesis and therapeutically inhibit inflammatory bone loss. MFG-E8 is expressed at high levels in certain pathologic conditions, such as chronic pancreatitis, obesity and tumorigenesis, but local administration of MFG-E8 in conditions with localized bone loss (e.g., periodontitis and rheumatoid arthritis) should not involve the undue risks required by systemic administration.

Described herein, BM-derived osteoclast precursors (OCPs) from Mfge8−/− mice underwent increased RANKL-induced osteoclastogenesis leading to enhanced resorption pit formation as compared with WT controls. Consistently, exogenously added MFG-E8 inhibited RANKL-induced osteoclastogenesis from mouse or human OCPs. Upon experimental periodontitis, an oral inflammatory disease characterized by loss of bone support of the dentition, Mfge8−/− mice exhibited higher numbers of osteoclasts and bone loss than WT controls. Accordingly, local microinjection of anti-MFG-E8 mAb exacerbated periodontal bone loss in WT mice. Conversely, microinjection of MFG-E8 inhibited inflammatory periodontal bone loss.

Therefore, MFG-E8 is a homeostatic regulator of osteoclasts and therapeutic compositions of MFG-E8 are useful to treat disorders associated with osteoinflammatory disorders, such as inflammatory bone loss.

### Compositions

One aspect of the invention includes a composition comprising an effective amount of milk fat globule-EGF factor 8 (MFG-E8) formulated for local administration and a pharmaceutically acceptable carrier or adjuvant.

The invention includes a composition formulated for oral administration, such as a liquid suspension, a chewable composition, and an orally disintegrating tablet or capsule composition. In one embodiment, the composition is formulated for delayed-release.

To avoid high systemic levels of MFG-E8 that may lead to certain pathologic conditions, such as chronic pancreatitis, obesity and tumorigenesis, the composition is formulated to target a site of bone loss, such as periodontal tissue, alveolar process, arthritic and non-arthritic joints, an injured bone, and other bone sites. In one embodiment, the composition comprises an effective amount in a range of about 0.1 g to about 2 μg per single dose. The effective amount includes a range of about 0.05 μg to about 150 μg per single dose, about 0.1 μg to about 100 μg, about 0.5 μg to about 50 μg, and about 1 μg to about 20 μg per single dose. The effective amount includes about 0.05 μg, 0.1 μg, 0.5 μg, 1 μg, 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 g, 150 μg, and any amount therebetweeen. In one embodiment, the effective amount includes a dosage of about 50 μg per human tooth.

In another embodiment, the composition further comprises at least one binder, excipient, diluent, or any combination thereof. In yet another embodiment, the composition further comprises at least one of an antimicrobial agent and an anti-inflammatory agent.

In another aspect, the invention includes a composition for treating a bone disorder comprising an effective amount of milk fat globule-EGF factor 8 (MFG-E8) formulated for local administration. In some embodiments, the effective amount inhibits at least one condition selected from the group consisting of osteoclastogenesis, inflammation and bone resorption.

### Methods

The present invention includes a method of regulating osteoclast activation and inhibiting bone loss at a target site comprising locally administering an effective amount of milk fat globule-EGF factor 8 (MFG-E8) to the target site.

In one embodiment, the administration inhibits expression of at least one osteoclast marker, such as NFATc1, cathepsin K, and αvβ3 integrin. In another embodiment, the administration inhibits osteoclastogenesis, such as RANKL-induced osteoclastogenesis. In yet another embodiment, the administration inhibits bone resorption. In still another embodiment, the administration inhibits expression and/or secretion of at least one bone resorption stimulator. The bone resorption stimulator includes, but is not limited to, TNF, IL-6, IL-17A, MMP-9, Ptgs2, RANKL, IL-17A, Tnfsf11, CXCL1, CXCL2, CXCL3, CXCL5, and combinations thereof. In another embodiment, the administration inhibits expression and/or secretion of at least one proinflammatory cytokine, such as IL-8 and CCL2/MCP-1.

Since the composition is locally administered, in some embodiments, the target site is selected from the group consisting of periodontal tissue, alveolar process, arthritic and non-arthritic joints, an injured bone, and other bone sites.

In another aspect, the invention includes a method of inhibiting inflammation at a target site comprising locally administering an effective amount of milk fat globule-EGF factor 8 (MFG-E8) to the target site. In one embodiment, the inhibition decreases expression of at least one inflammation molecule selected from the group consisting of a pro-inflammatory mediator, an adhesion molecule and an immune receptor, such as IL-6, IL-8, IL-17a, MMP9, PTGS2, TNFSF11, SPP1, CSF3, CXCL1, CXCL2, CXCL3, CXCL5, ITGAL, SELE, CXCR2, CCR1, and TREM1.

In another embodiment, the target site is selected from the group consisting of periodontal tissue, alveolar process, arthritic and non-arthritic joints, an injured bone, and other bone sites. In yet another embodiment, the target site is the periodontal tissue and the administration is in a gingival tissue. Under these administration conditions, periodontal microbiota growth is also suppressed.

In yet another aspect, the invention includes a method of treating a bone disorder comprising local administration of an effective amount of milk fat globule-EGF factor 8 (MFG-E8) to a target site. The target site includes, but is not limited to, any site affected by a bone disorder, such as periodontal tissue, alveolar process, an arthritic and non-arthritic joint, an injured bone, bone in need of bone growth, bone in need of bone regeneration, and any other bone site. In one embodiment, the bone disorder is selected from the group consisting of osteoporosis, osteomalacia, osteosclerosis, and osteopetrosis.

The therapeutic method of regulating osteoclast activation at a target site includes inhibiting bone loss at a target site, inhibiting inflammation at a target site, and treating a bone disorder in a subject. In another embodiment, the therapeutic method includes administering a therapeutically effective amount of milk fat globule-EGF factor 8 (MFG-E8) to a subject (e.g., animal, human) in need thereof, where the subject includes a mammal, particularly a human. Such treatment is suitably administered to a subject, particularly a human, suffering from, having, susceptible to, or at risk for developing a bone disorder or a symptom thereof.

### Pharmaceutical Compositions and Formulations

The invention also encompasses pharmaceutical composition of the invention for therapeutic methods of treatment. In one aspect, the invention includes use of a composition comprising an effective amount of milk fat globule-EGF factor 8 (MFG-E8) formulated for local administration and a pharmaceutically acceptable carrier or adjuvant for treating a bone disorder, such as osteoporosis, osteomalacia, osteosclerosis, and osteopetrosis. In one embodiment, the pharmaceutical composition is provided in a form suitable for a particular route of administration to a subject, and includes at least one binder, excipient, diluent, or any combination thereof. In another embodiment, the composition of the invention comprises an antimicrobial agent, an anti-inflammatory agent, and a combination thereof.

Pharmaceutical compositions that are useful in the methods of the invention may be suitably developed for intragingival, inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, low dosage intravenous and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that are useful in the present invention are not limited to the particular formulations and compositions that are described herein.

Other contemplated formulations include projected nanoparticles, liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In one embodiment, the compositions of the invention are formulated using one or more pharmaceutically acceptable excipients or carriers. In one embodiment, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

### Administration/Dosing

In the clinical settings, delivery systems for the therapeutic composition can be introduced into a patient by any of a number of methods, each of which is familiar in the art. For instance, a pharmaceutical composition can be administered orally, e.g. by tablet or oral suspension. In other embodiments, initial delivery of the composition is more limited with introduction into the animal being quite localized. For example, the composition can be introduced by catheter (see U.S. Pat. No. 5,328,470) or by stereotactic injection (e.g. Chen, et al. PNAS 91: 3054-3057 (1994)).

The regimen of administration may affect what constitutes an effective amount. The therapeutic formulations may be administered to the patient either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or condition in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods.

The following working examples therefore, specifically point out the preferred embodiments of the present invention, and are not to be construed as limiting in any way the remainder of the disclosure.

The Results of the experiments disclosed herein are now described.

It was discovered that MFG-E8 is expressed by and regulates osteoclasts (OCLs), giant multinucleated cells (MNCs) that resorb bone during normal bone remodeling and also under pathologic inflammatory conditions (e.g., rheumatoid arthritis and periodontitis). OCLs differentiate from BM precursors in the monocyte/macrophage lineage. In this process, M-CSF promotes the survival and proliferation of osteoclast precursors which are induced to express RANK, thereby becoming competent to respond to RANKL, a key cytokine for OCL differentiation and activation. The findings described herein indicate that MFG-E8 is a novel regulator that restrains RANKL-induced osteoclast differentiation and function and can be used therapeutically to inhibit inflammatory bone loss.

### Osteoclasts (OCLs) Express MFG-E8.

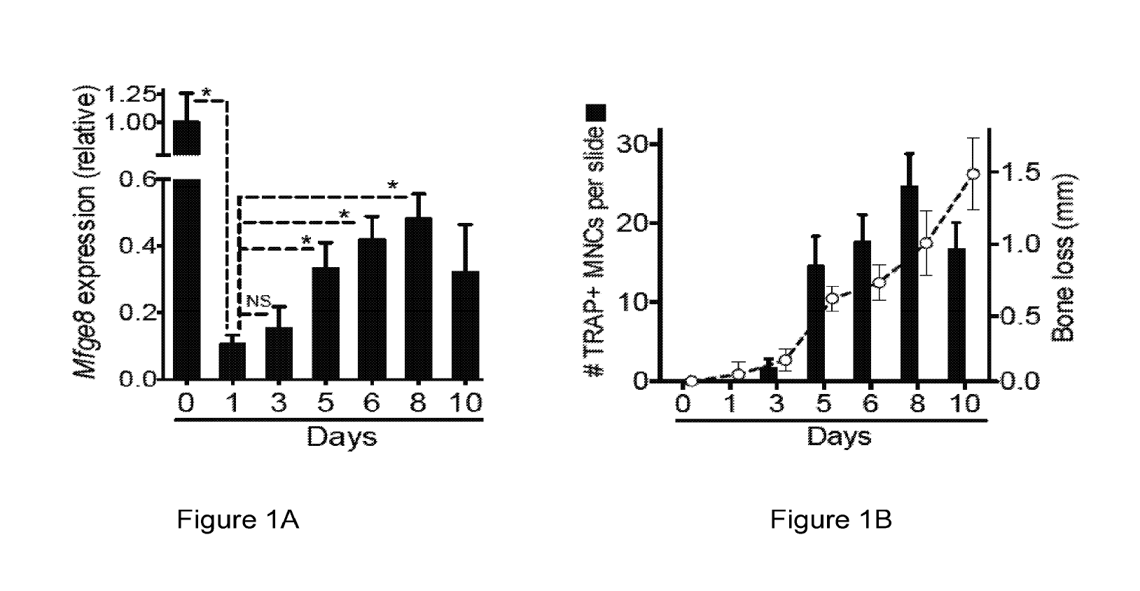

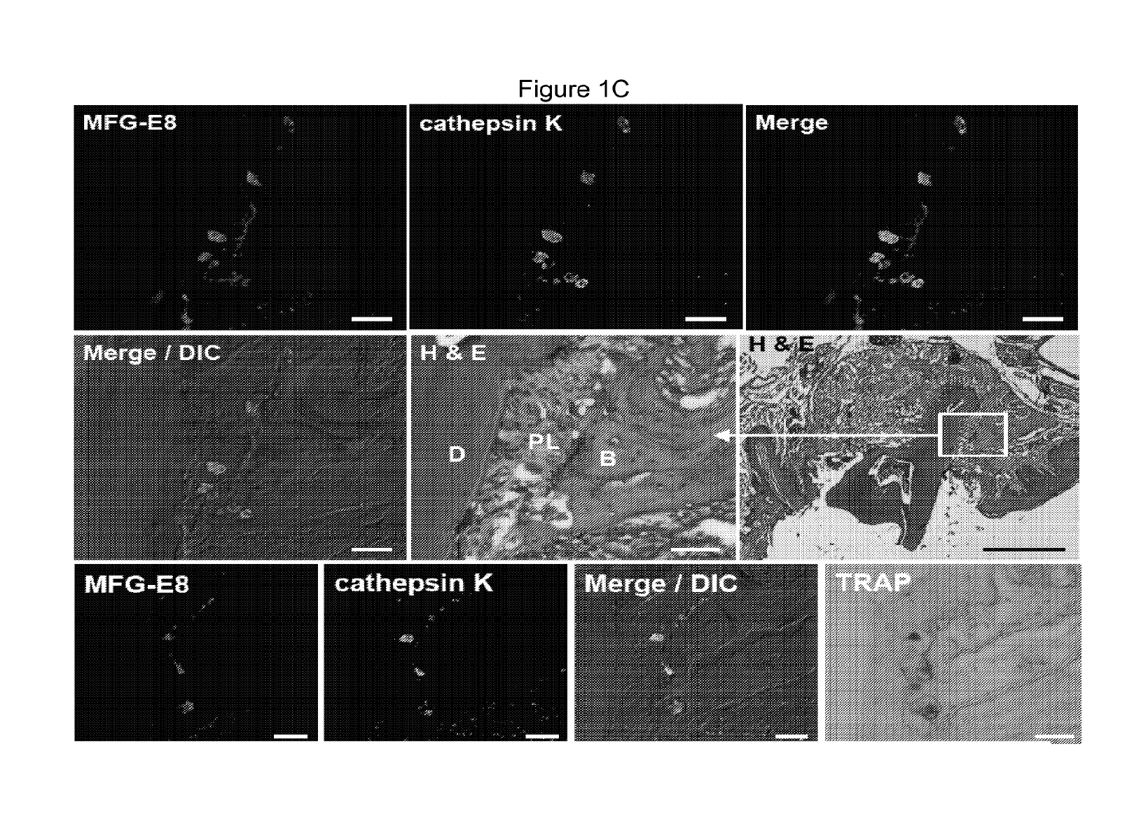

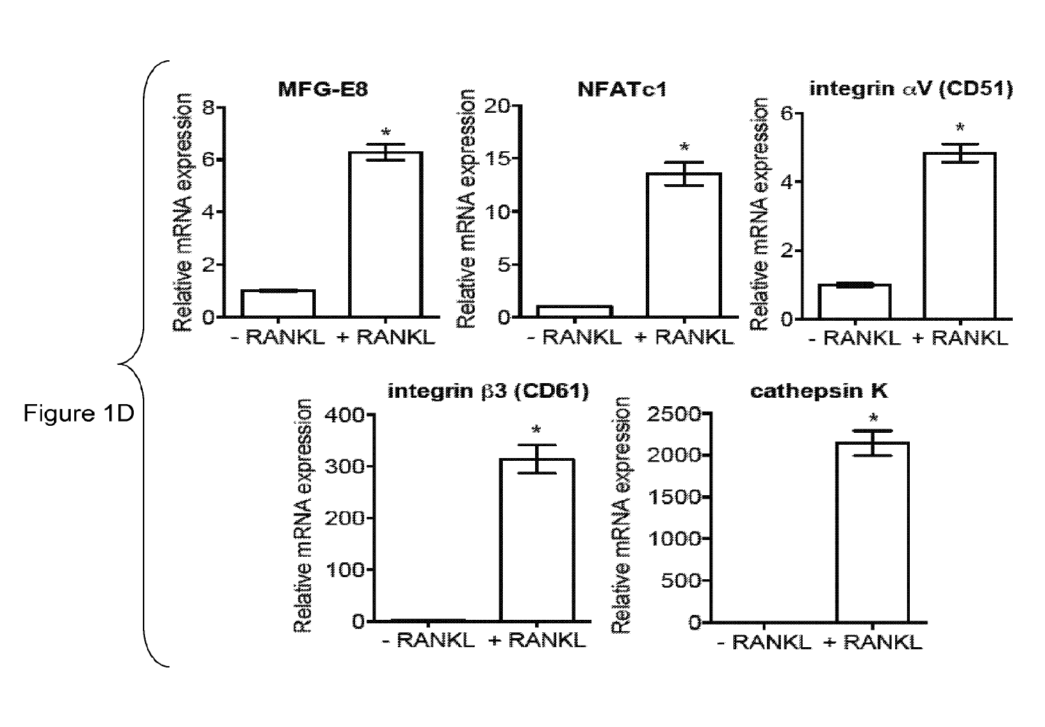

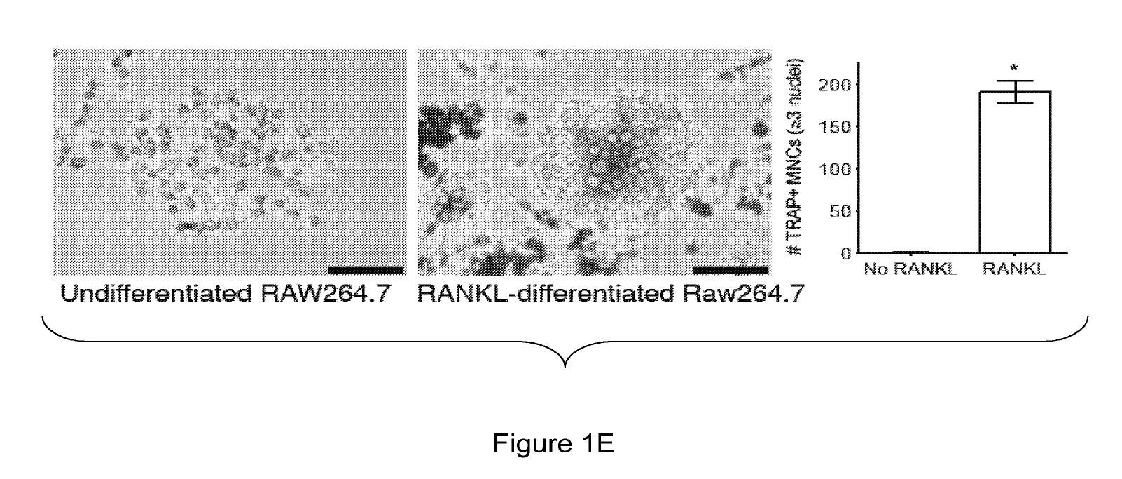

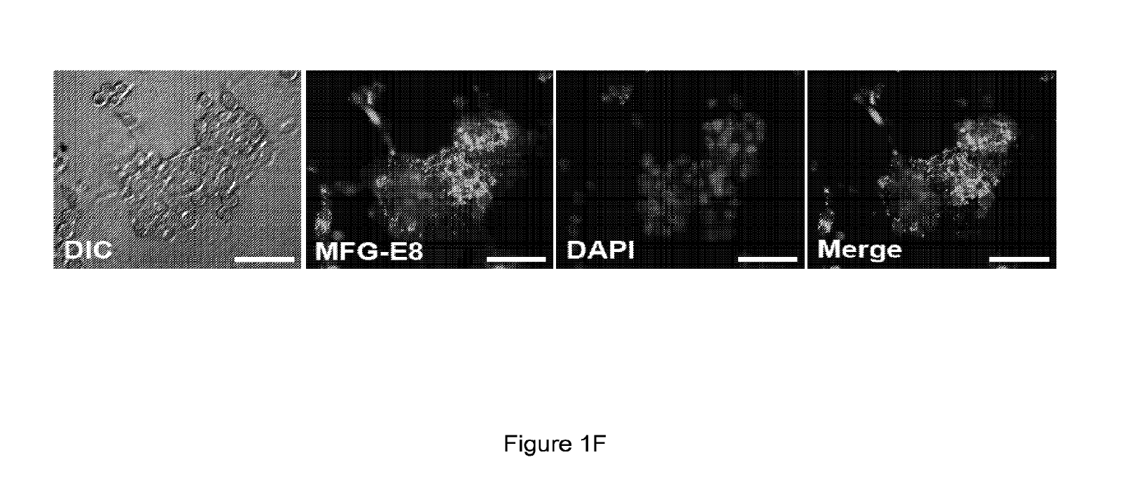

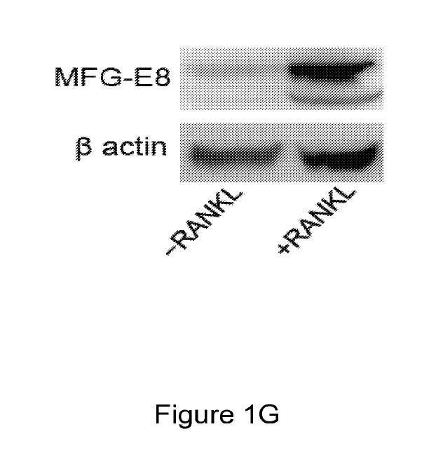

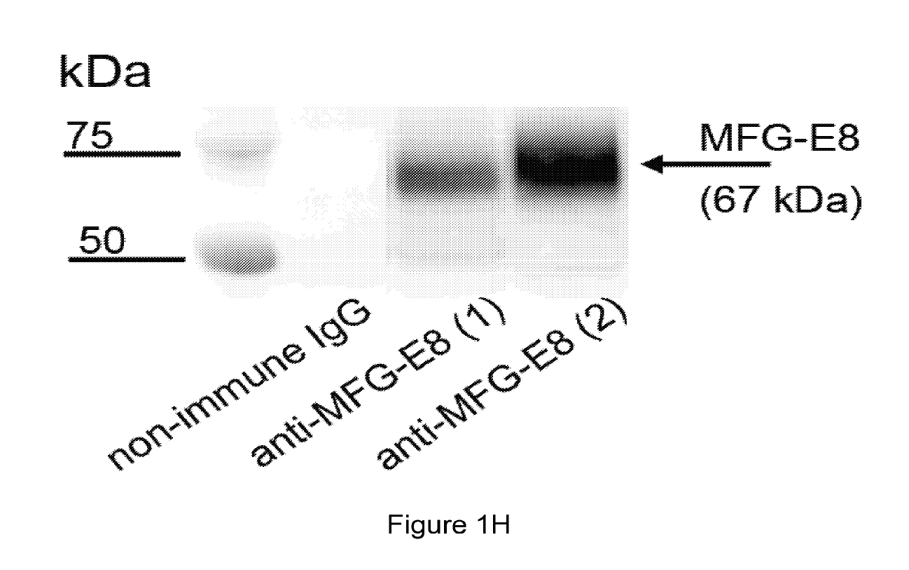

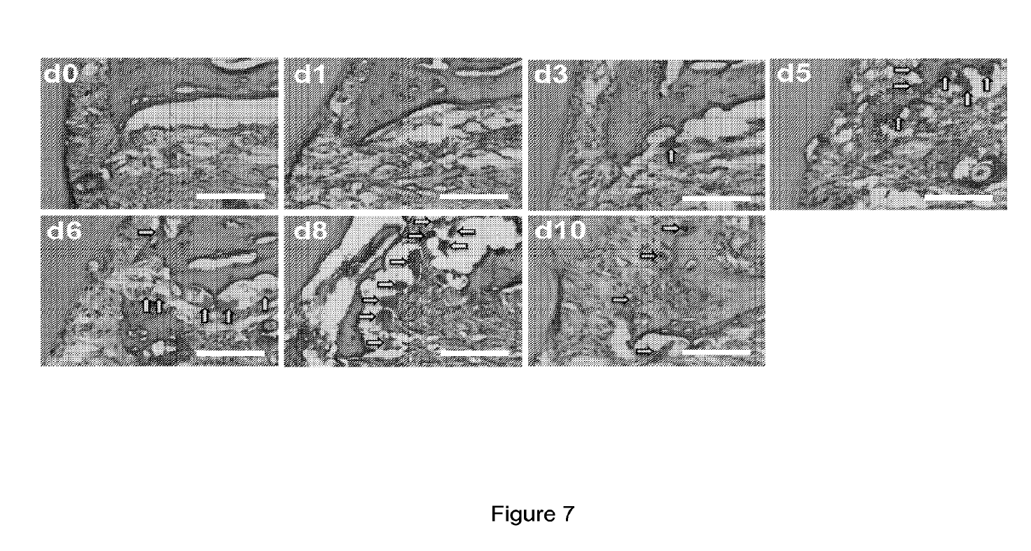

In view of the anti-inflammatory potential of MFG-E8, the role of MFG-E8 was determined in periodontitis, a microbiota-induced inflammatory disease causing loss of bone support of the dentition. Using the ligature-induced periodontitis model in mice, the expression of MFG-E8 mRNA in the periodontal tissue was monitored. Consistent with MFG-E8 downregulation seen in other models of inflammation, periodontal MFG-E8 mRNA levels were dramatically decreased upon placement of the ligatures (from dO to dl; FIG. 1A). Subsequently, and unexpectedly, MFG-E8 mRNA expression gradually increased until d8 (FIG. 1A). The resurgence of MFG-E8 expression correlated with the appearance of OCLs, the numbers of which increased to d8 but dropped on d10 (FIG. 1B and FIG. 7), at which time MFG-E8 expression also appeared to decline (FIG. 1A). This correlation suggested that MFG-E8 might derive from OCLs in the course of periodontitis (being a cumulative process, bone loss continued to rise to d10; FIG. 1B, right Y axis).

Consistent with this notion, MFG-E8 was detected in regions of cathepsin K expression, at the interface of connective tissue (periodontal ligament) and bone (FIG. 1C). The sites of MFG-E8 and cathepsin K expression coincided with tartrate-resistant acid phosphatase (TRAP)+ cells (FIG. 1C; bottom row), further strengthening the notion that OCLs express MFG-E8.

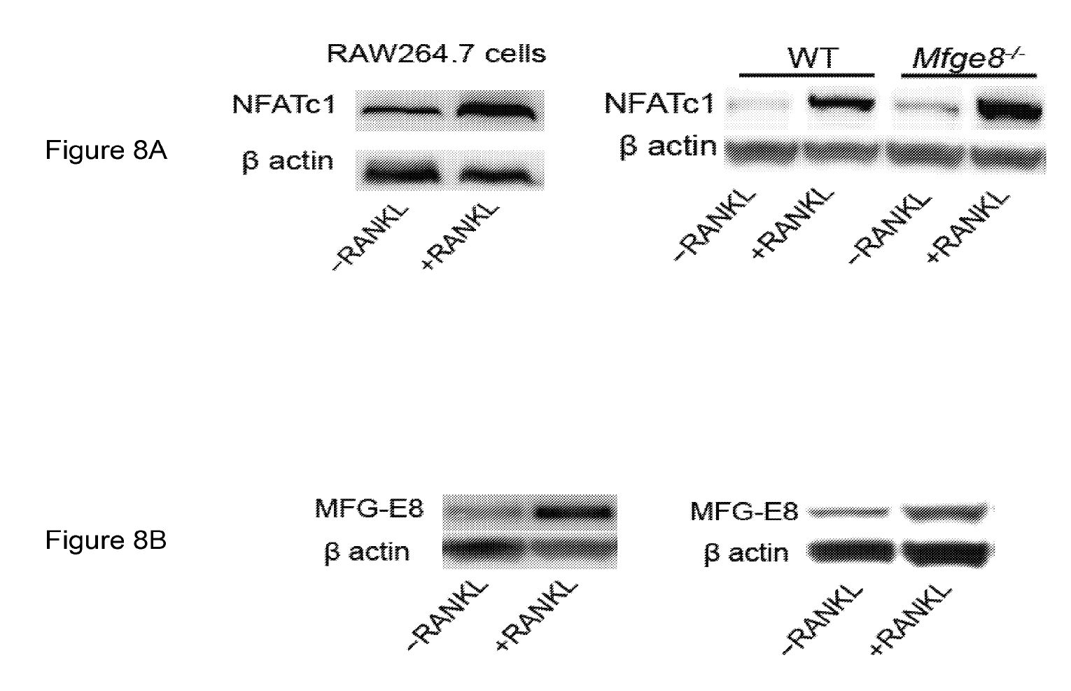

RAW264.7 cells are widely used as a model for OCL differentiation and function, as RANKL-induced RAW264.7 gene expression and developmental and functional characteristics are similar to those of OCLs in vivo or OCLs generated in vitro from primary precursor cells. Consistent with the concept that OCLs may constitute a source of MFG-E8, we showed that RANKL-differentiated RAW264.7 OCLs express MFG-E8 mRNA (six-fold upregulation as compared to undifferentiated RAW264.7 cells), in addition to established activation and functional markers, such as NFATc1, the heterodimeric αvβ3 integrin (CD51/CD61), and cathepsin K (FIG. 1D). NFATc1, the master transcription factor for OCL differentiation was also upregulated at the protein level (FIG. 8A). The generated OCLs (identity confirmed by expression of typical differentiation markers and morphologically after TRAP staining in FIGS. 1D and 1E) also expressed MFG-E8 protein, as shown by cell immunofluorescence (FIG. 1F), immunoblotting of cell lysates (FIG. 1G), and immunoprecipitation from culture supernatants (FIG. 1H). MFG-E8 protein expression, determined by whole-cell lysate immunoblotting, was also shown for primary OCLs generated from mouse BM-derived precursors or from human CD14+ monocytes (FIG. 8B). Taken together, the in vivo and in vitro observations show for the first time that osteoclasts express MFG-E8.

### MFG-E8 Regulates Osteoclast Differentiation and Function.

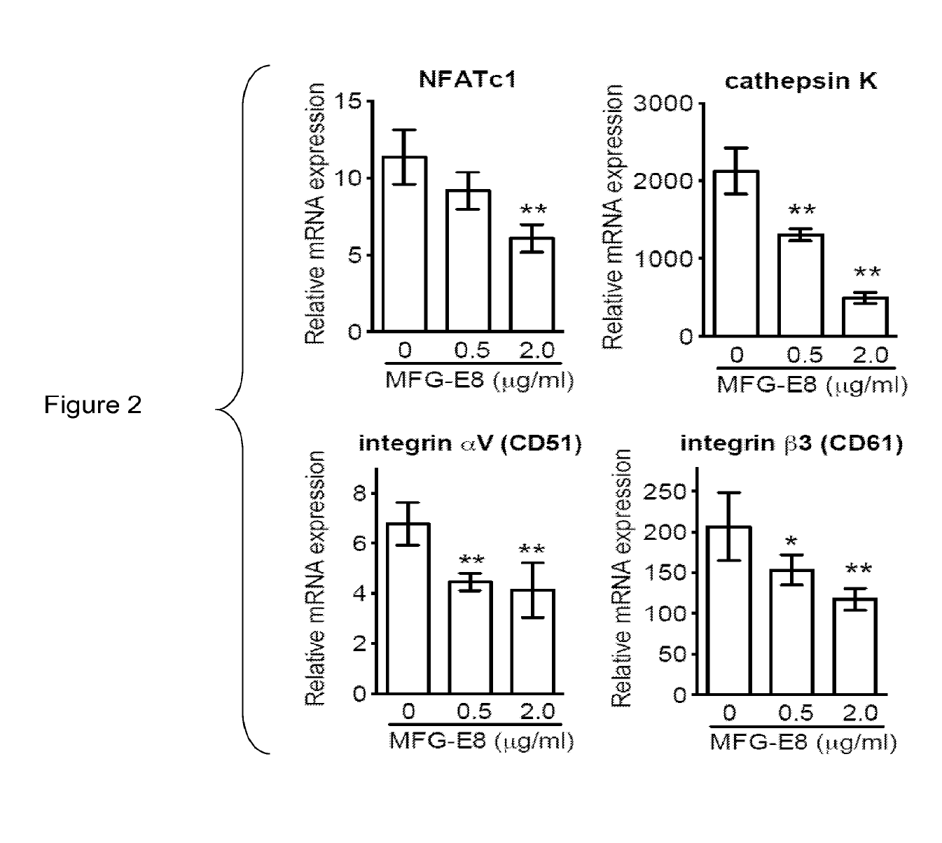

To characterize the role of MFG-E8 in osteoclastogenesis, the differentiation of RANKL-stimulated RAW264.7 cells in the absence or presence of exogenously added recombinant (r)MFG-E8 was examined. rMFG-E8 inhibited the expression of OCL activation and functional markers, namely, NFATc1, the master transcription factor for OCL differentiation, cathepsin K, and the heterodimeric αvβ3 integrin (CD51/CD61) (FIG. 2).

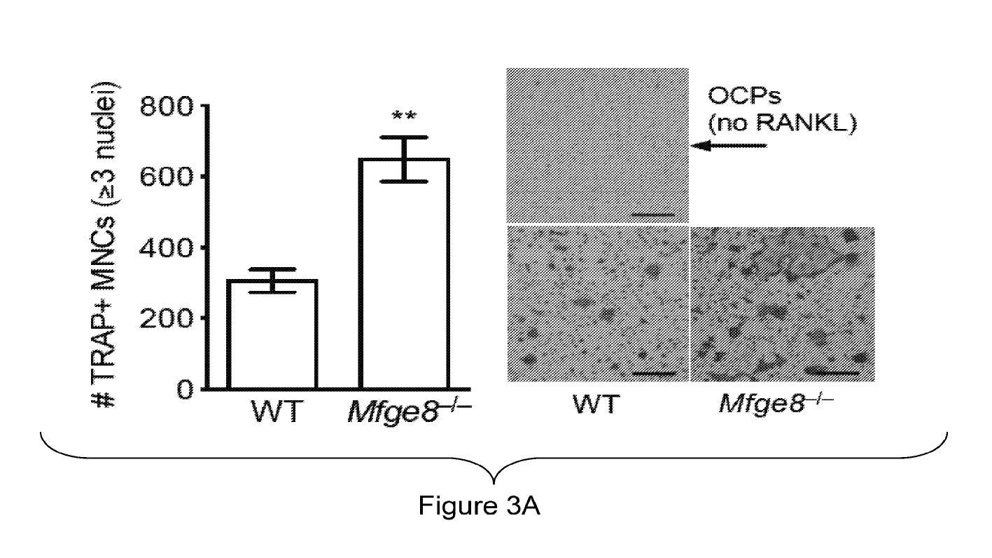

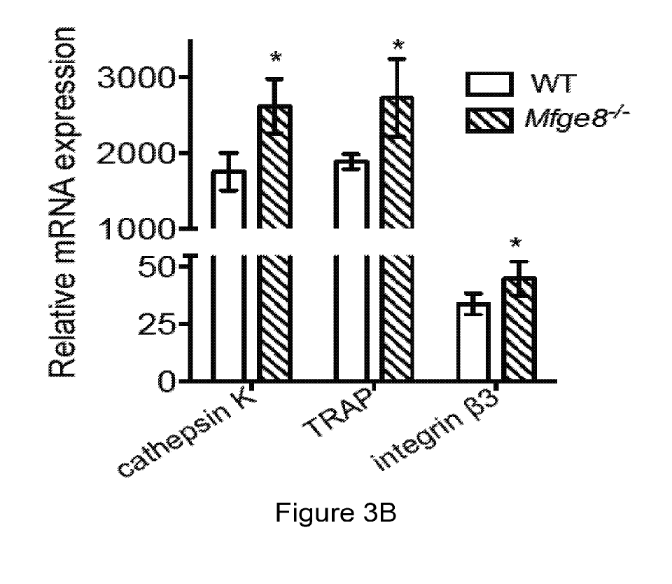

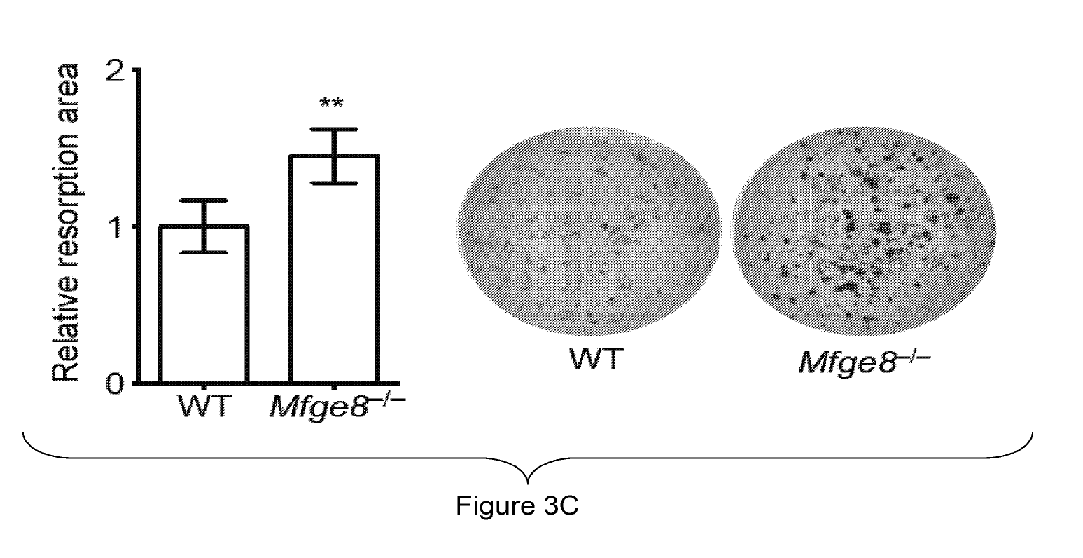

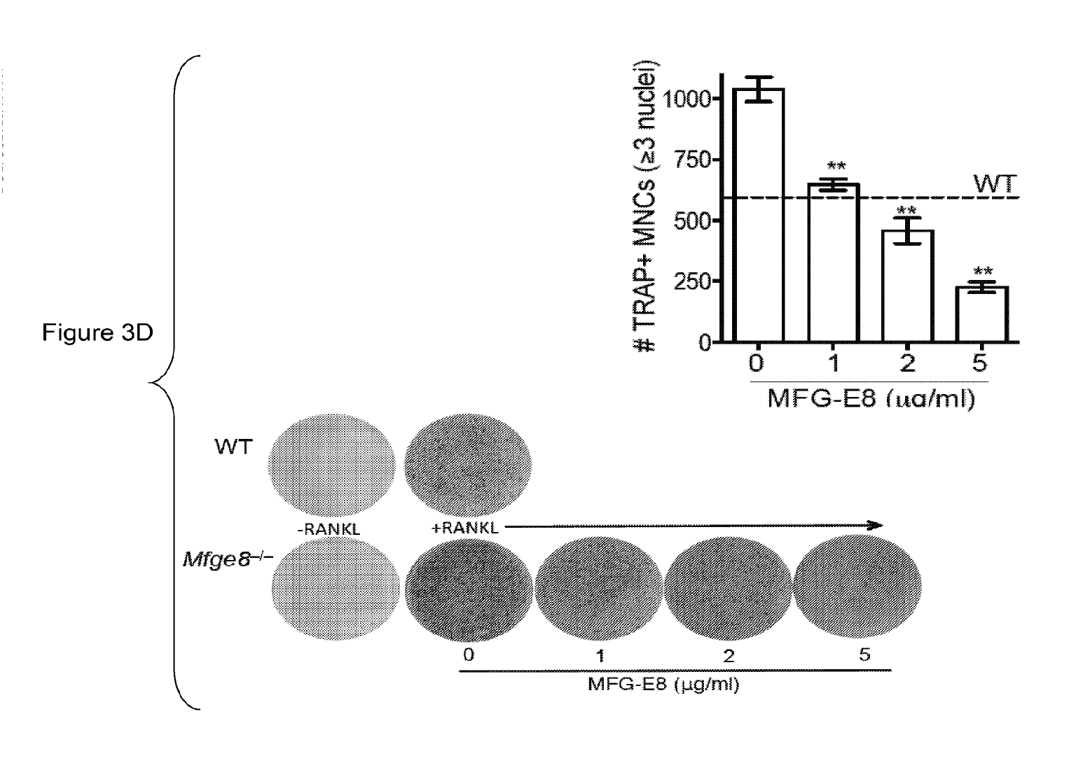

To obtain conclusive evidence that MFG-E8 is involved in homeostatic regulation of OCLs, OCLs from WT or MFG-E8-deficient (Mfge8−/−) osteoclast precursors (OCP) were generated from BM. Mfge8−/−OCPs underwent more efficient osteoclastogenesis (higher numbers of TRAP+MNCs) than WT OCPs (FIG. 3A), consistent with higher expression of OCL markers (cathepsin K, TRAP, and integrin β3) (FIG. 3B).

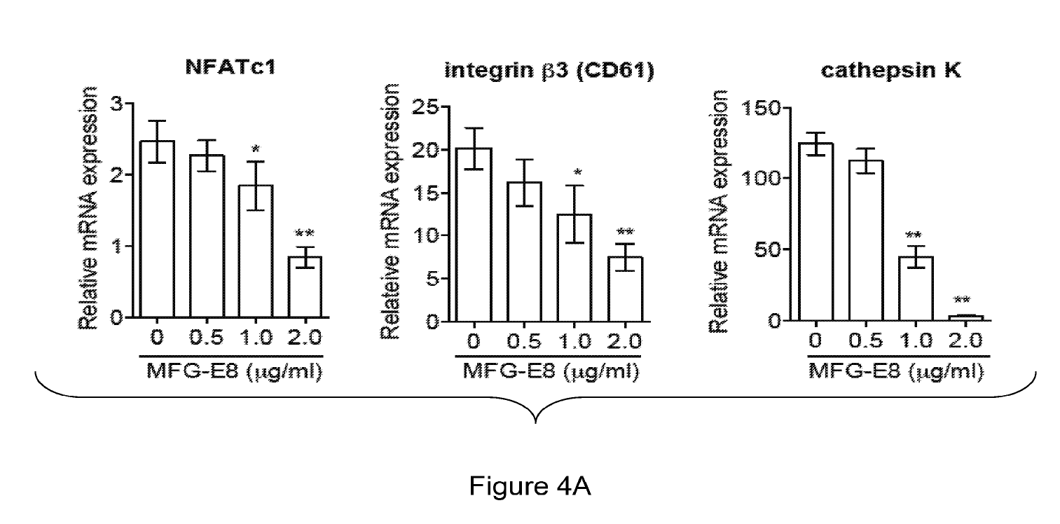

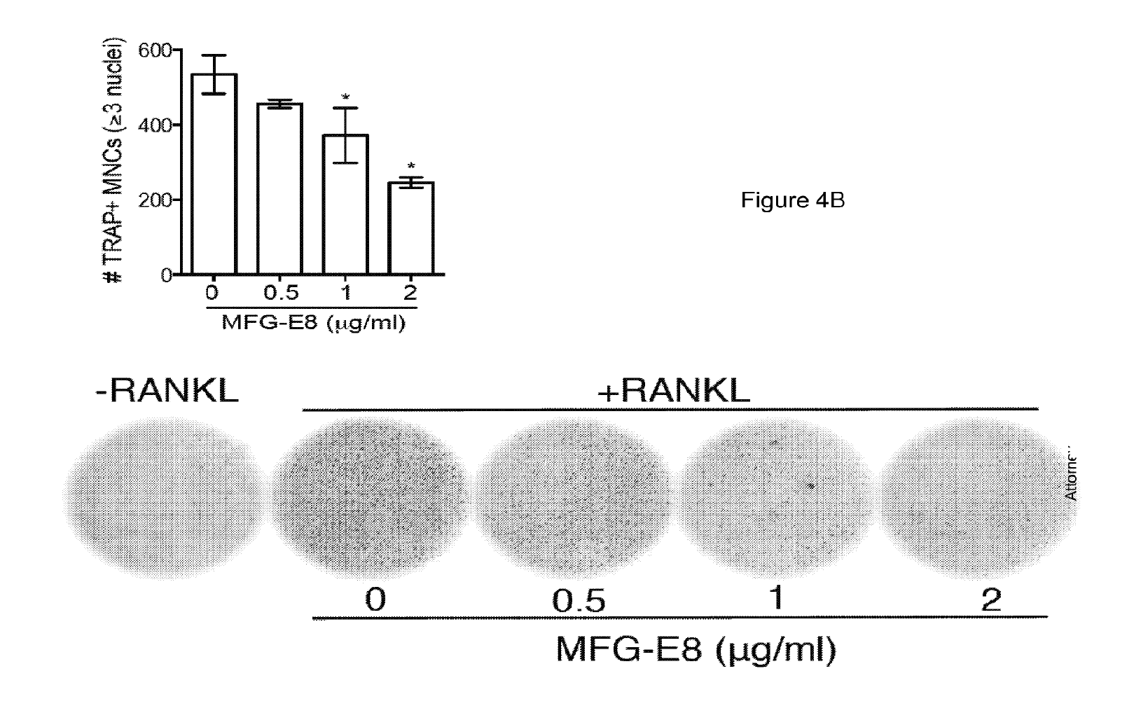

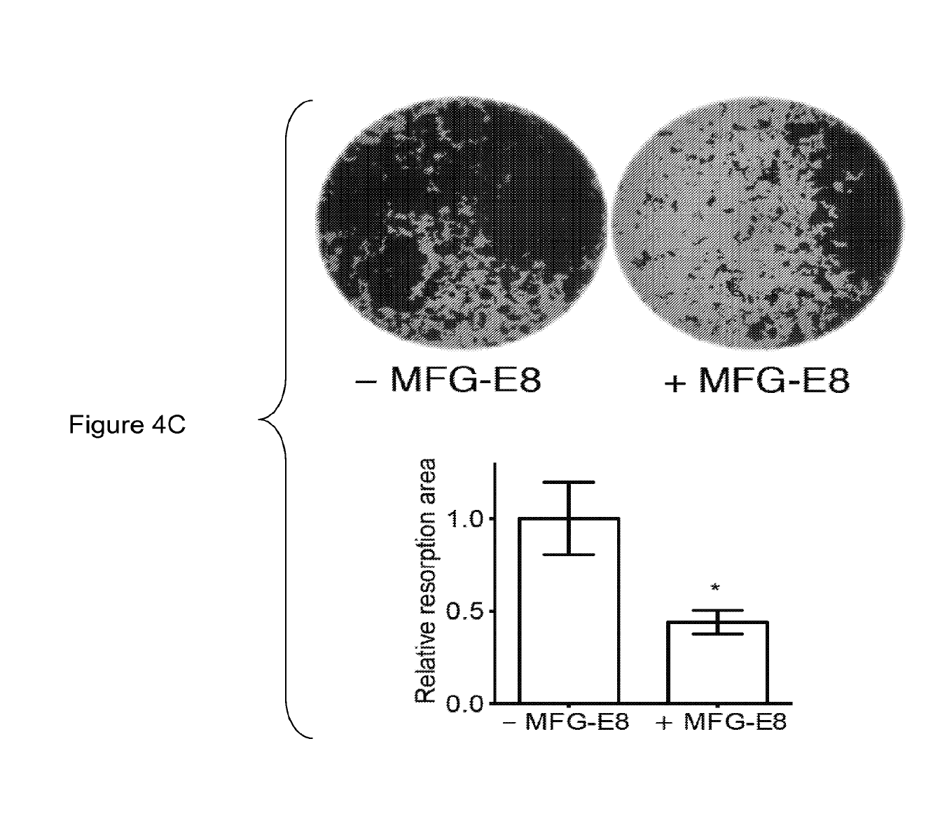

Moreover, Mfge8−/−OCLs caused enhanced resorption pit formation as compared with their WT counterparts (FIG. 3C). Importantly, addition of rMFG-E8 inhibited osteoclastogenesis from Mfge8−/−OCP in a dose-dependent manner (FIG. 3D). The degree of Mfge8−/− osteoclastogenesis in the presence of 1-2 μg/ml MFG-E8 was comparable to WT osteoclastogenesis (FIG. 3D). These data implicate MFG-E8 as a novel negative regulator of osteoclastogenesis, at least in mice. MFG-E8 may have a similar function in humans. Indeed, in a system of osteoclastogenesis from human CD14+ monocytes, human rMFG-E8 inhibited RANKL-induced expression of OCL differentiation and functional markers (FIG. 4A), osteoclastogenesis (FIG. 4B), and resorption pit formation (FIG. 4C).

MFG-E8 Deficiency is Associated with Increased Osteoclastogenesis and Bone Loss in Vivo.

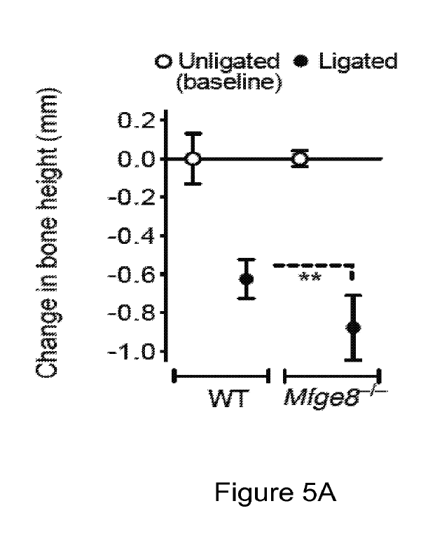

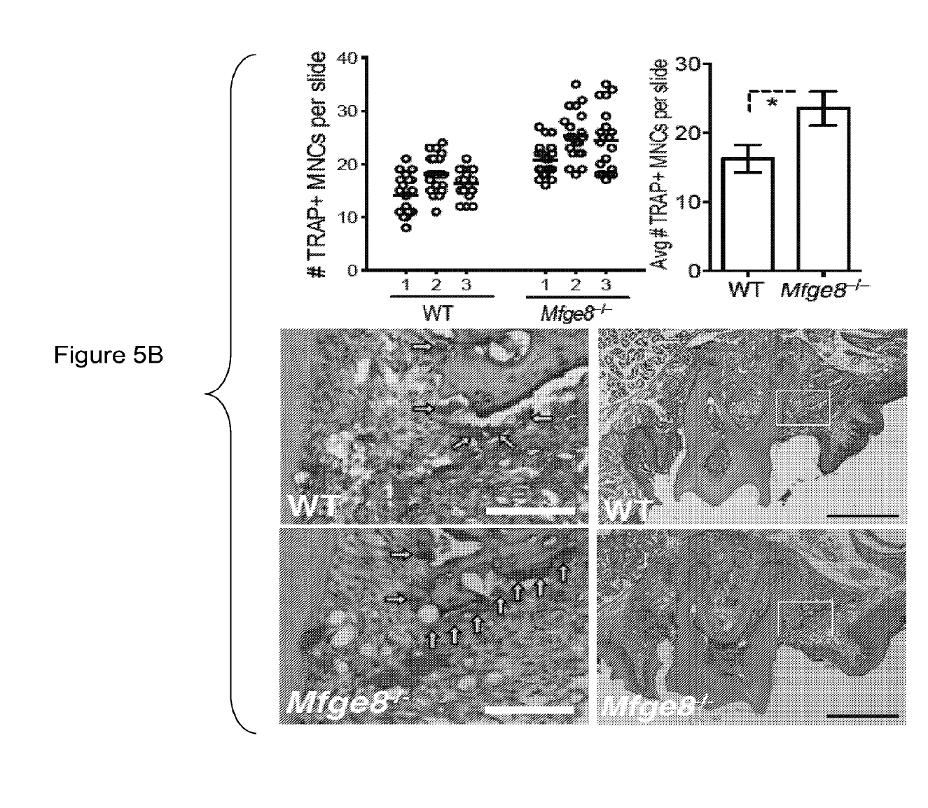

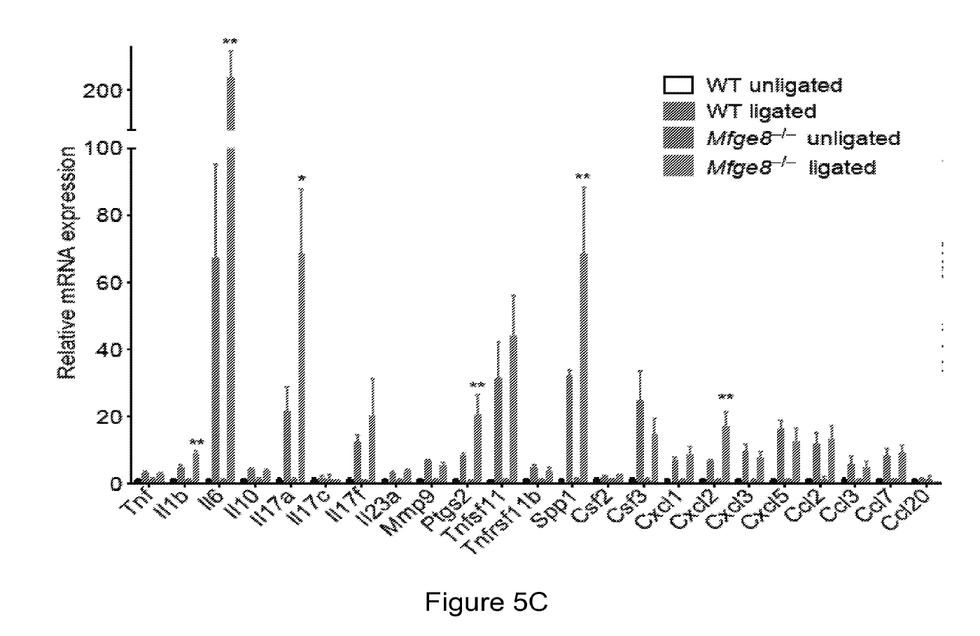

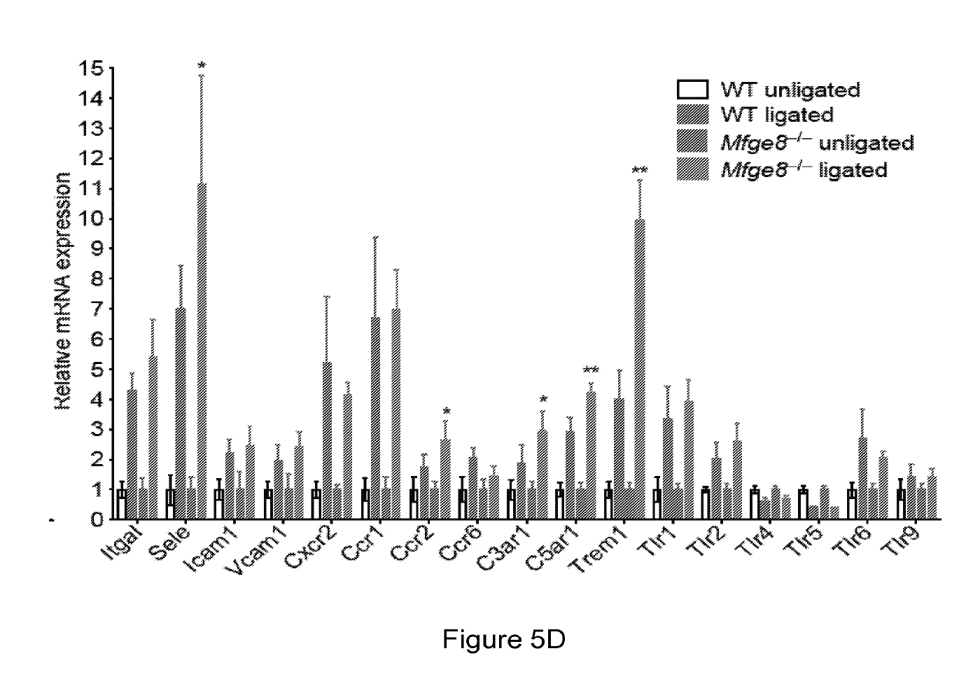

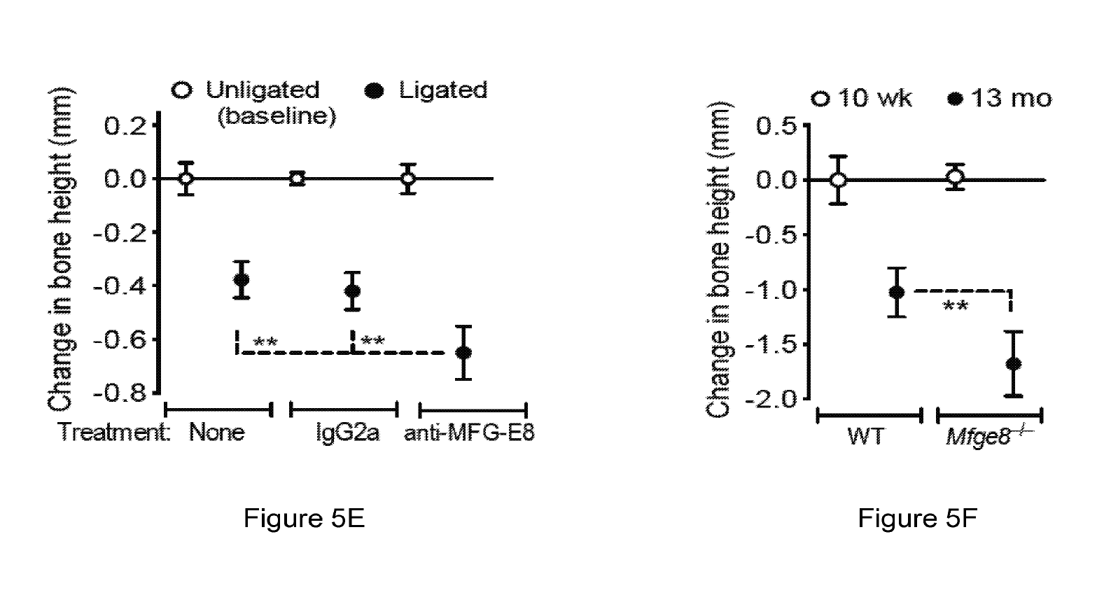

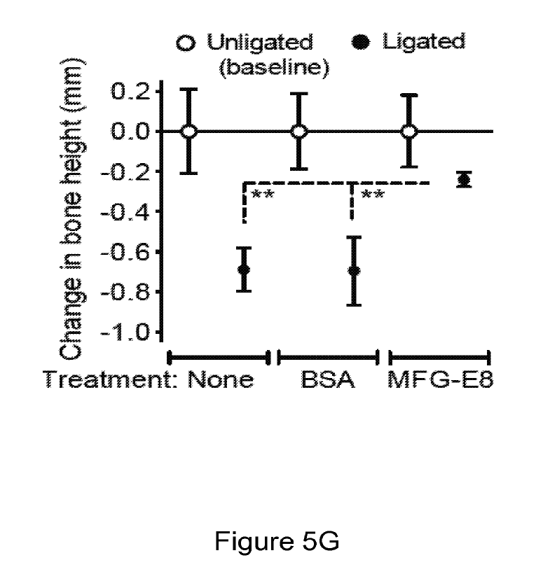

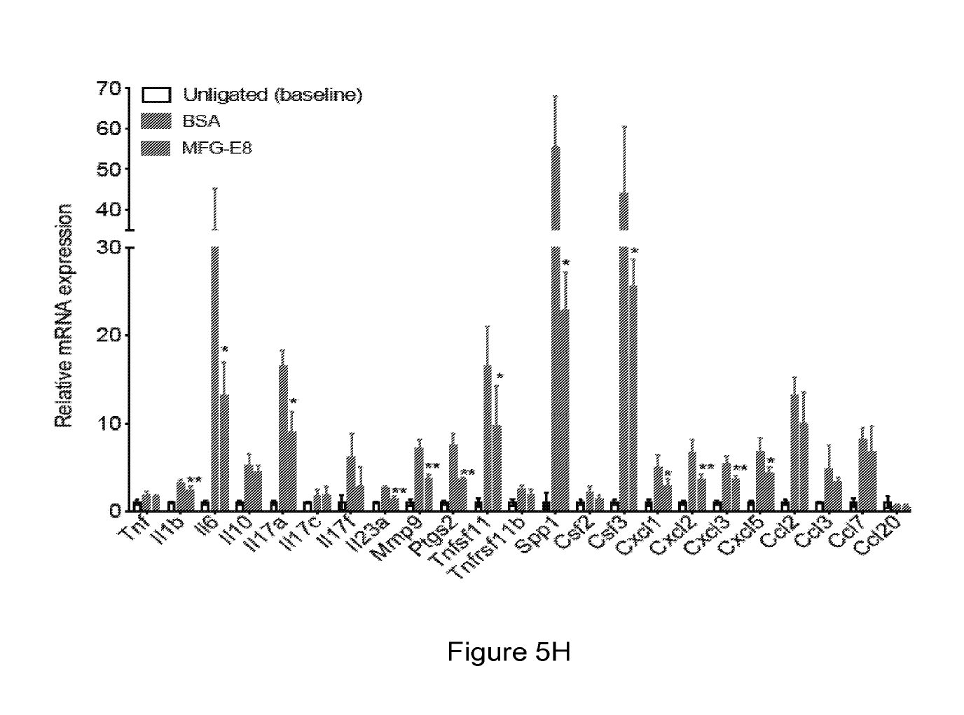

To test the importance of MFG-E8 in in vivo osteoclastogenesis and bone loss, WT and Mfge8−/− mice were subjected to ligature-induced periodontitis. Mfge8−/− mice exhibited significantly more bone loss (FIG. 5A) and higher numbers of OCLs in the periodontal tissue (FIG. 5B) than WT controls. In both WT and Mfge8−/− mice, ligature-induced periodontitis elevated the expression of inflammatory mediators as well as adhesion molecules and innate immune receptors, although the differences between the two experimental groups reached statistical significance for a subset of the genes examined (FIGS. 5C and 5D). Interestingly, whereas Mfge8−/− mice displayed significantly higher expression of certain bone-resorption-promoting molecules (e.g., IL-17A and osteopontin [Sppl]) compared with WT mice, the two groups had comparable expression of RANKL (Tnfsf11) and its natural inhibitor, osteoprotegerin (Tnfrsf11b) (FIG. 5C), suggesting that the in vivo anti-osteoclastogenic effect of endogenous MFG-E8 may not involve alterations in RANKL expression.

Local gingival microinjection of an anti-MFG-E8 mAb (but not isotype control) significantly enhanced ligature-induced bone loss as compared to control mice (FIG. 5E), further supporting the importance of endogenous MFG-E8 in bone homeostasis. Aging mice, like aging humans, develop naturally-occurring chronic periodontal bone loss (21, 22).

To determine the role of MFG-E8 in this process, Mfge8−/− mice were raised in parallel with WT controls. Although 10-wk-old Mfge8−/− and WT mice had comparable bone heights, by the age of 13 mo Mfge8−/− mice experienced significantly more bone loss than age matched WT mice (FIG. 5F). Taken together, these data conclusively implicate MFG-E8 as a negative regulator of bone loss in vivo.

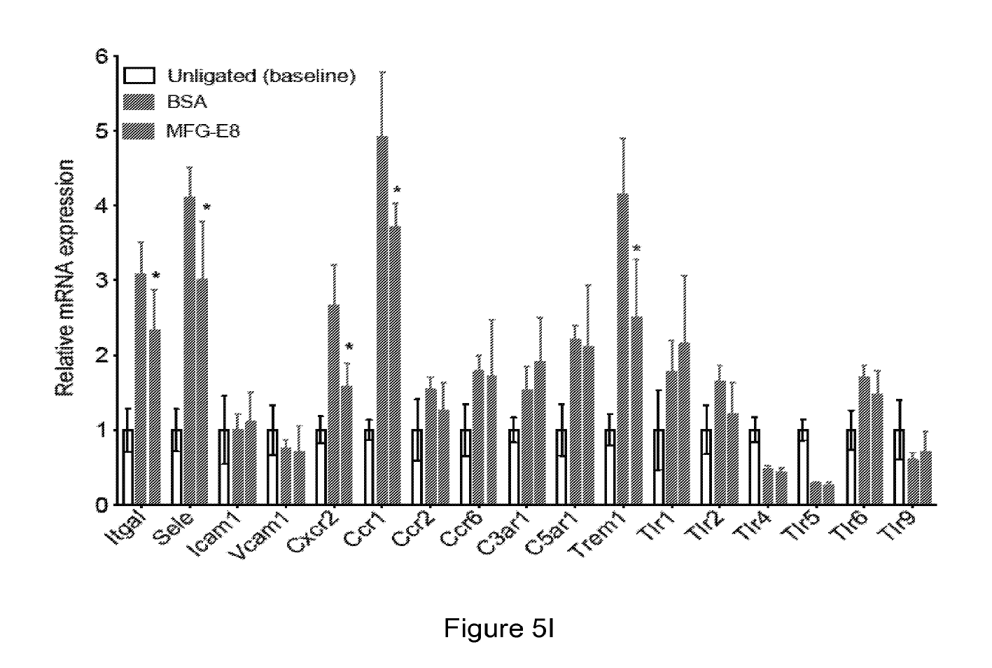

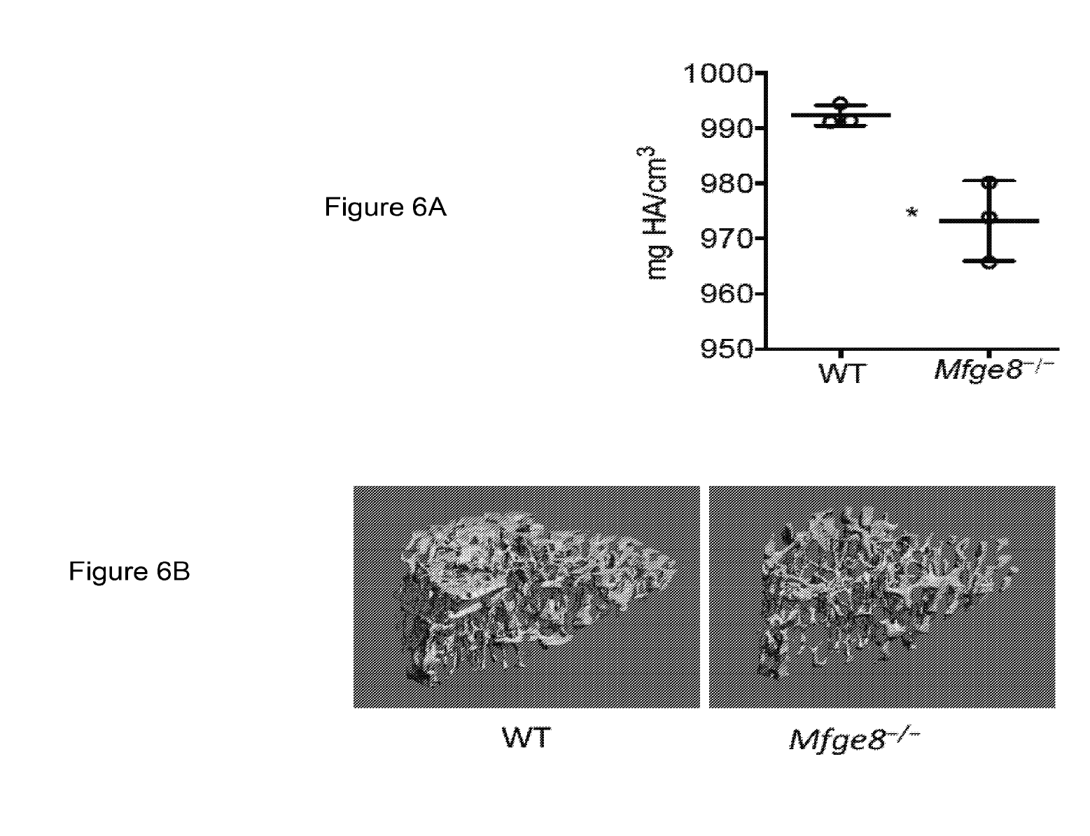

Moreover, μCT analysis showed a modest but statistically significant reduction in the tissue mineral density of tibiae of Mfge8^{-/-} mice as compared to WT controls (FIGS. 6A and 6B). This finding suggests that MFG-E8 might also regulate bone mass in the absence of an inflammatory condition.

### Local Administration of MFG-E8 Inhibits Inflammation and Bone Loss In Vivo.

It was determined whether rMFG-E8 could be exploited therapeutically to inhibit bone loss upon ligature-induced periodontitis in WT mice. Local microinjection of rMFG-E8 into the gingival significantly inhibited bone loss as compared to untreated control mice, whereas similar treatment with BSA failed to protect against bone loss (FIG. 5G). Mice treated with rMFG-E8 also exhibited decreased mRNA encoding several pro-inflammatory mediators as well as adhesion molecules and innate immune receptors relative to BSA-treated mice (FIGS. 5H and 5I). This finding is consistent with the reported anti-inflammatory action of MFG-E8, which may have contributed to inhibition of bone loss.

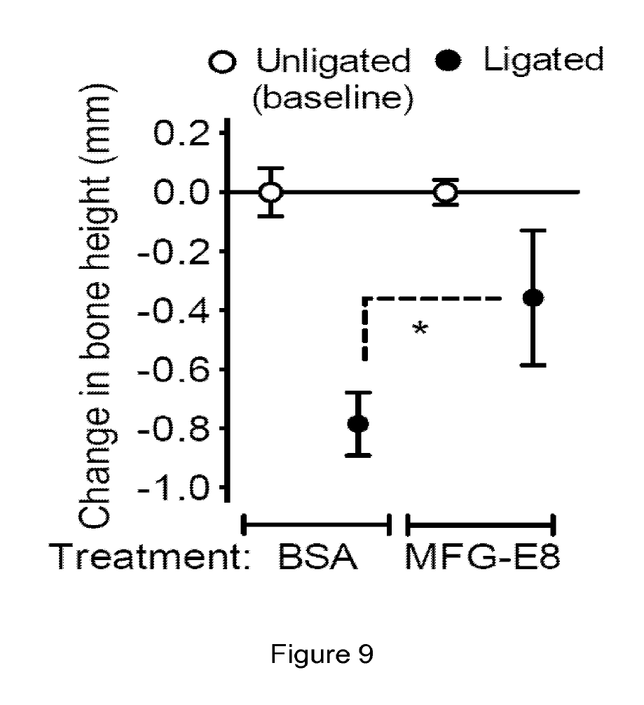

Similar to its effect in WT mice, rMFG-E8 also protected Mfge8−/− mice against ligature-induced bone loss (FIG. 9).

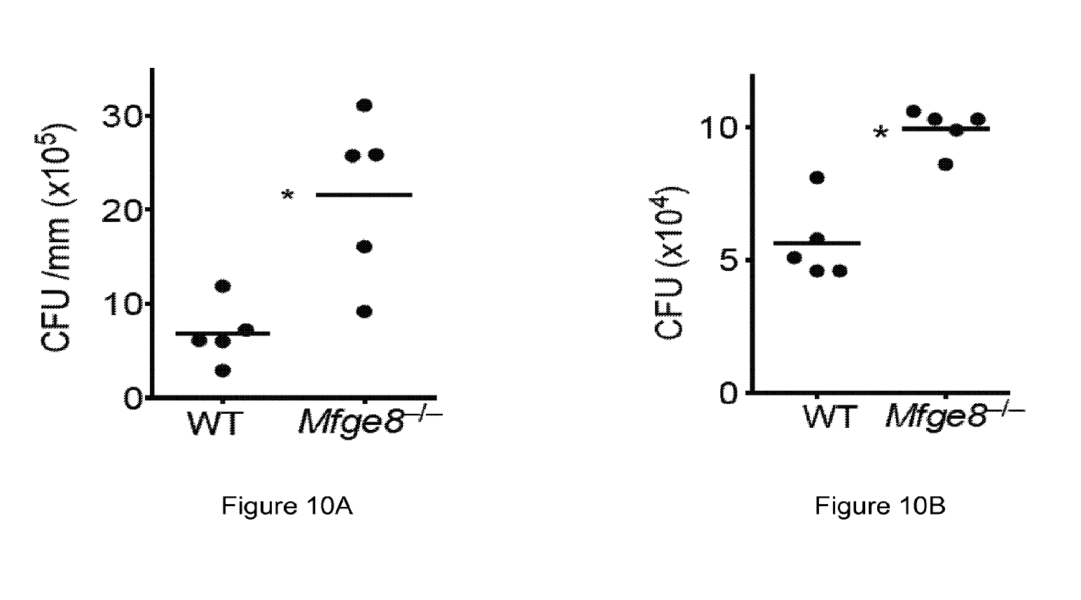

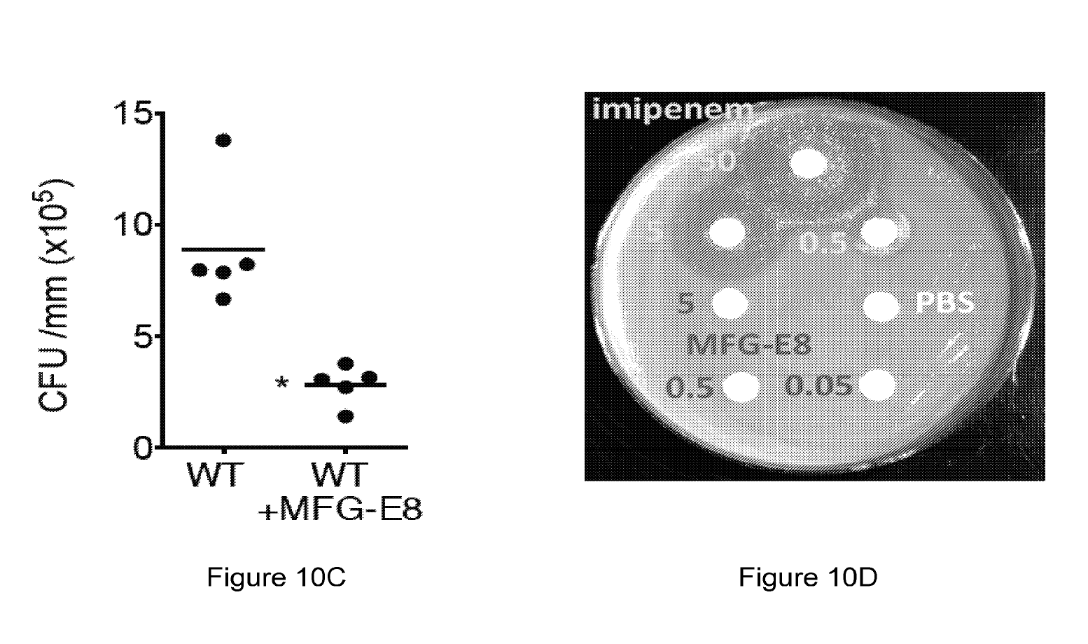

Intriguingly, MFG-E8 deficiency was associated with increased periodontal bacterial burden and, accordingly, treatment of WT mice with rMFG-E8 significantly decreased the bacterial load (FIG. 10A-10C). In disk inhibition zone assays with numerous bacterial isolates from murine periodontal tissue, rMFG-E8 failed to exert direct killing activity, in contrast to the antibiotic imipenem (control) (FIG. 10D). Therefore, the suppressive effect of MFG-E8 on the periodontal microbiota is likely mediated by its capacity to inhibit inflammation, and thus limits growth of periodontal bacteria that utilize tissue breakdown products (e.g., peptides and heme-containing compounds). In conclusion, autocrine MFG-E8 regulates OCL homeostasis in vitro and in vivo.

### DISCUSSION

Homeostatic mechanisms are of paramount importance to the proper functioning of any biological system. OCLs rely on several modulators to control their function and the findings indicate that MFG-E8 is one of them. This novel modulator is upregulated during osteoclastogenesis, in line with most biological systems where negative regulators are upregulated to control functional activity and prevent pathological states. The importance of MFG-E8 in restraining or fine-tuning osteoclast differentiation and function is highlighted by the effects of its absence: OCPs from Mfge8−/− mice underwent increased RANKL-induced osteoclastogenesis leading to enhanced resorption pit formation and, consistently, Mfge8^{-/-} mice experienced more periodontal bone loss than WT controls. In both cases, administration of exogenous MFG-E8 reversed the overactive phenotype, thereby preventing excessive osteoclastic activity in vitro and ligature-induced periodontal bone loss in vivo.

It is conceivable that OCLs produce MFG-E8 at levels that would control but not abrogate their differentiation and function, whereas therapeutic doses of MFG-E8 can have stronger inhibitory effects that could effectively diminish pathologic bone resorption. This notion is consistent with the dose-dependent inhibitory effects of MFG-E8 in FIG. 3D: At 1-2 μg/ml, exogenously added MFG-E8 simply restrained osteoclastogenesis from Mfge8^{-/-}OCPs, rendering it comparable to WT osteoclastogenesis (i.e., in the presence of endogenously produced MFG-E8). At a higher concentration (5 μg/ml), MFG-E8 diminished osteoclastogenesis (>75% inhibition). It thus appeared that endogenously produced MFG-E8 acted homeostatically to restrain unwarranted osteoclastogenesis, although from a therapeutic standpoint higher concentrations of exogenously added MFG-E8 could inhibit this process further.

Not surprisingly, the differences between WT and Mfge8^{-/-} osteoclastogenesis and resorption pit formation were relatively modest despite being statistically and biologically significant, especially in an in vivo setting. Indeed, given adequate time, 13-month-old Mfge8^{-/-} mice experienced>60% more periodontal bone loss than age-matched WT controls. In this regard, aging mice, like aging humans, develop naturally-occurring chronic periodontal bone loss, and the results suggested that endogenously produced MFG-E8 was an important regulator of this process.

The observations for decreased expression of proinflammatory cytokines and chemokines in the periodontal tissue of MFG-E8-treated mice undergoing ligature-induced periodontitis (as compared with BSA-treated controls) were consistent with the reported anti-inflammatory action of MFG-E8. A possible anti-inflammatory mechanism of MFG-E8 involves its ability to interfere and prevent downstream activation of inflammatory mediators. It has been shown that inflammatory mediators including IL-1β, IL-6, and IL-17 play important roles in periodontal inflammation and bone loss. The ability of MFG-E8 to inhibit the expression of these proinflammatory molecules as described herein, suggested an indirect way by which MFG-E8 down-regulated osteoclastogenesis and bone loss. Whereas the anti-inflammatory and anti-osteoclastogenic of MFG-E8 can be readily dissociated and investigated separately in vitro, the strong connection between inflammation and osteoclastogenesis suggested that the therapeutic application of MFG-E8 was capable of a two-pronged attack on periodontitis and perhaps other inflammatory bone disorders.

The placement of ligatures induces bacteria-mediated inflammation in the periodontal tissue and this may explain the observed initial downregulation of MFG-E8 expression (about 90% reduction from day 0 to day 1), in line with similar observations in other models of inflammation. The subsequent resurgence of MFG-E8 expression correlated temporally and spatially with osteoclastogenesis in the course of ligature-induced periodontitis. Since in vitro formed OCLs expressed and were regulated by MFG-E8, it was likely that the reappearance of MFG-E8 in the course of experimental periodontitis was contributed (at least in part) by the generated OCLs, ostensibly to regulate their differentiation and function. This notion is consistent with the findings that Mfge8^{-/-} mice experience increased osteoclastogenesis and periodontal bone loss as compared to WT controls.

The findings on the indirect antimicrobial effects of MFG-E8 added to accumulating evidence that inhibition of periodontal inflammation exerted a negative impact on the periodontal microbiota. In this regard, inflammation generated tissue breakdown products (e.g., peptides and heme-containing compounds) that could serve as critical nutritional needs of periodontal bacteria. Conversely, and consequently, the control of inflammation would be expected to limit bacterial growth, thereby explaining the inhibitory effects of MFG-E8 on the periodontal microbiota despite lacking intrinsic antimicrobial activity.

In summary, autocrine MFG-E8 regulated OCL homeostasis and rMFG-E8 could be a new therapeutic platform for the treatment of bone loss disorders. The anti-inflammatory action of MFG-E8 can further contribute to the control of bone loss in inflammatory conditions. Whereas diminished expression of MFG-E8 is associated with certain inflammatory diseases, MFG-E8 is expressed at high levels and is implicated in the pathogenesis of certain other pathologic conditions, such as chronic pancreatitis, obesity, and tumorigenesis (in humans and/or animal models). Therefore, caution is required in future MFG-E8-based therapeutic strategies, although the local administration of MFG-E8 in conditions with localized bone loss (e.g., periodontitis and rheumatoid arthritis) should not involve undue risk.

### MFG-E8 Expression in Human Cells

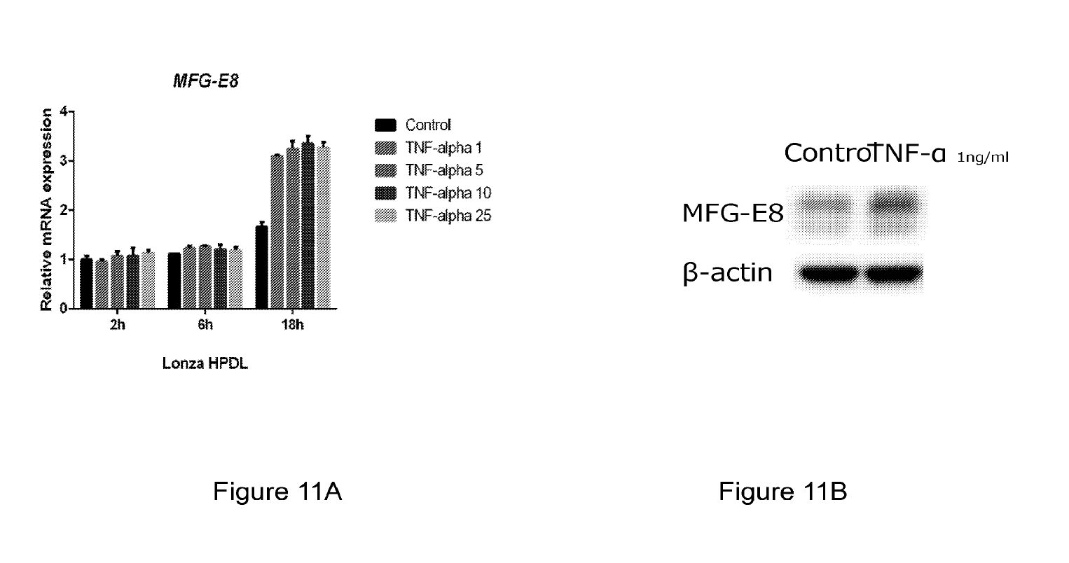

MFG-E8 expression was assessed in human periodontal ligament (HPDL) cells after stimulation with TNF-α. FIGS. 11A and 11B show, respectively, that MFG-E8 mRNA and protein expression in human periodontal ligament cells (HPDL) was increased after TNF-α stimulation.

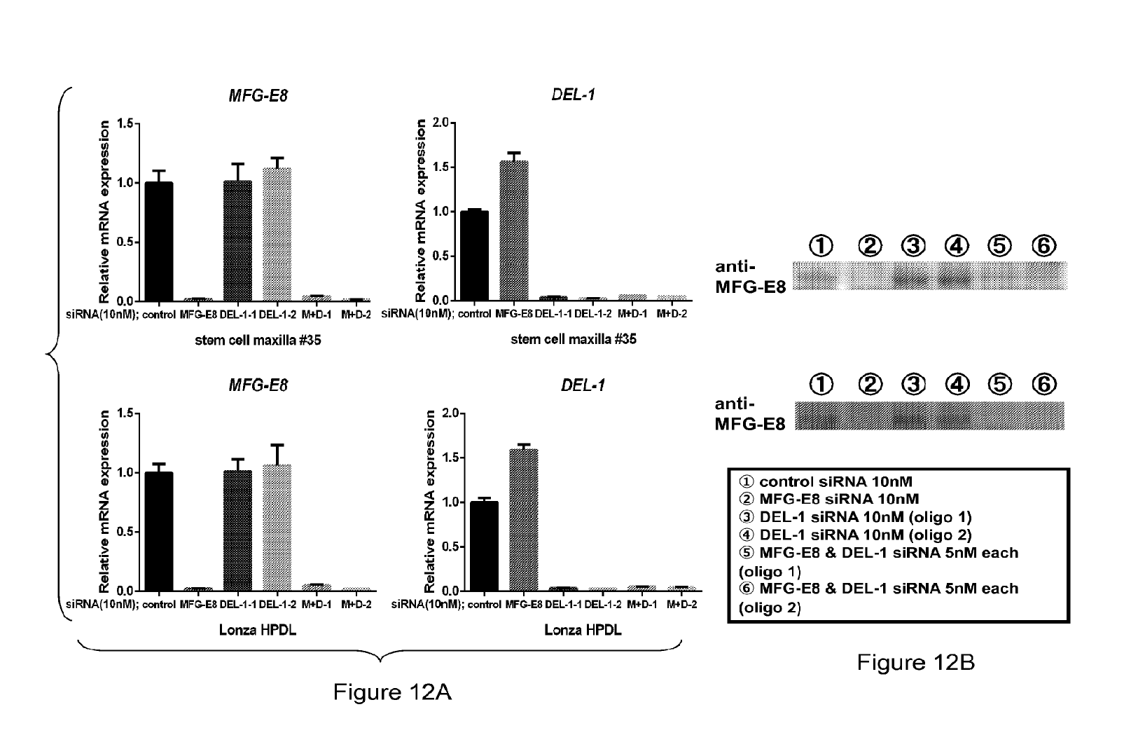

Silencing of MFG-E8 was analyzed by after siRNA introduction to either MFG-E8 or DEL-1 in maxilla stem cells or human periodontal ligament cells. FIG. 12A shows the specificity of MFG-E8 siRNA to silence endogenous MFG-E8 expression in both cell types (left graphs). Further, DEL-1 siRNA silenced endogenous DEL-1 expression in stem cell maxilla and human periodontal ligament cells (right graphs) without affecting MFG-E8 expression. FIG. 12B further demonstrates the effectiveness of the siRNAs on MFG-E8 and DEL-1 expression in stem cell maxilla (upper blot) and human periodontal ligament (lower blot) cells.

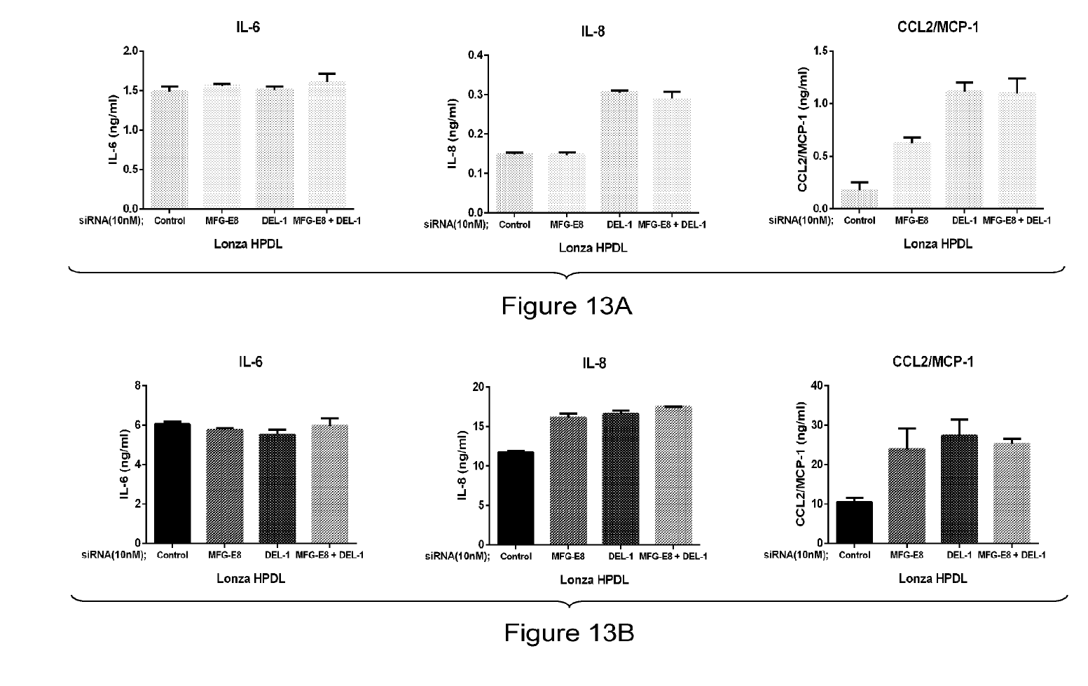

Levels of secreted factors, IL-6, IL-8 and CCL2/MCP-1, were assessed in HPDL cells after TNF-α stimulation when MFG-E8 expression was silenced. FIGS. 13A and 13B show that IL-8 levels and CCL2/MCP-1 in HPDLs without or with TNF-α stimulation (upper and lower panels, respectively) were increased in HPDL cells expressing MFG-E8 siRNA and stimulated with TNF-α, whereas IL-8 levels were decreased in HPDL cells expressing MFG-E8 siRNA and stimulated with TNF-α. These data indicate that endogenous MFG-E8 and Del-1 inhibit the expression of the proinflammatory cytokines, IL-8 and CCL2/MCP-1, in HPDLs at steady state or under inflammatory conditions.

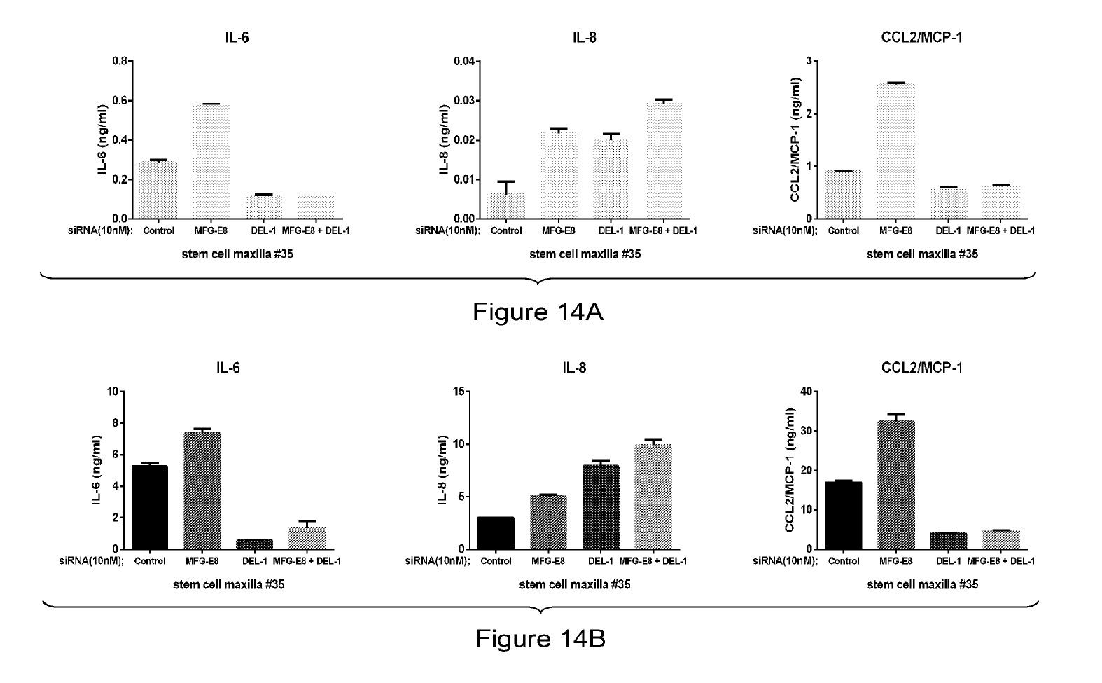

FIG. 14 shows that inhibition of expression of endogenous MFG-E8 results in increased expression of IL-8 and CCL2/MCP-1 in maxilla stem cells without or with TNF-α stimulation (upper and lower panels, respectively). These data indicate that endogenous MFG-E8 inhibits the expression of the proinflammatory cytokines IL-8 and CCL2/MCP-1 in in maxilla stem cells at steady state or under inflammatory conditions.

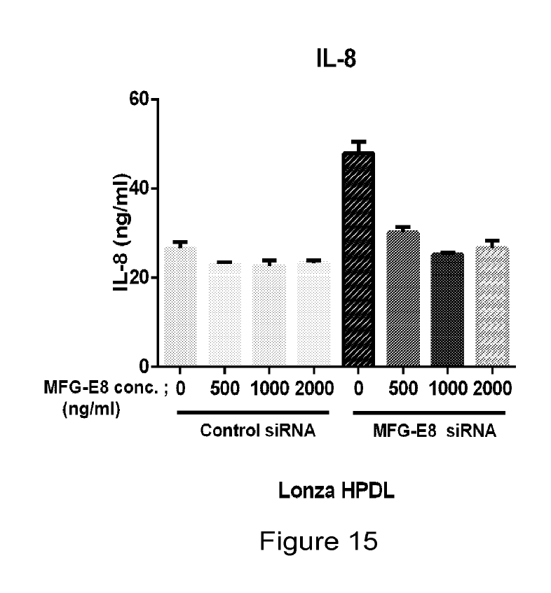

IL-8 levels were restored to normal concentrations when HPDL cells were treated with rhMFG-E8 after TNF-α stimulation and MFG-E8 siRNA, see FIG. 15. FIG. 15 shows that the ability of MFG-E8-specific siRNA to increase the expression of IL-8 in HPDLs is counteracted by exogenous addition of rhMFG-E8, thus confirming that effect of MFG-E8-specific siRNA was mediated through inhibition of endogenous MFG-E8 expression.

### MFG-E8 as a Fusion Fc Protein Blocks Periodontitis in Non-Human Primates

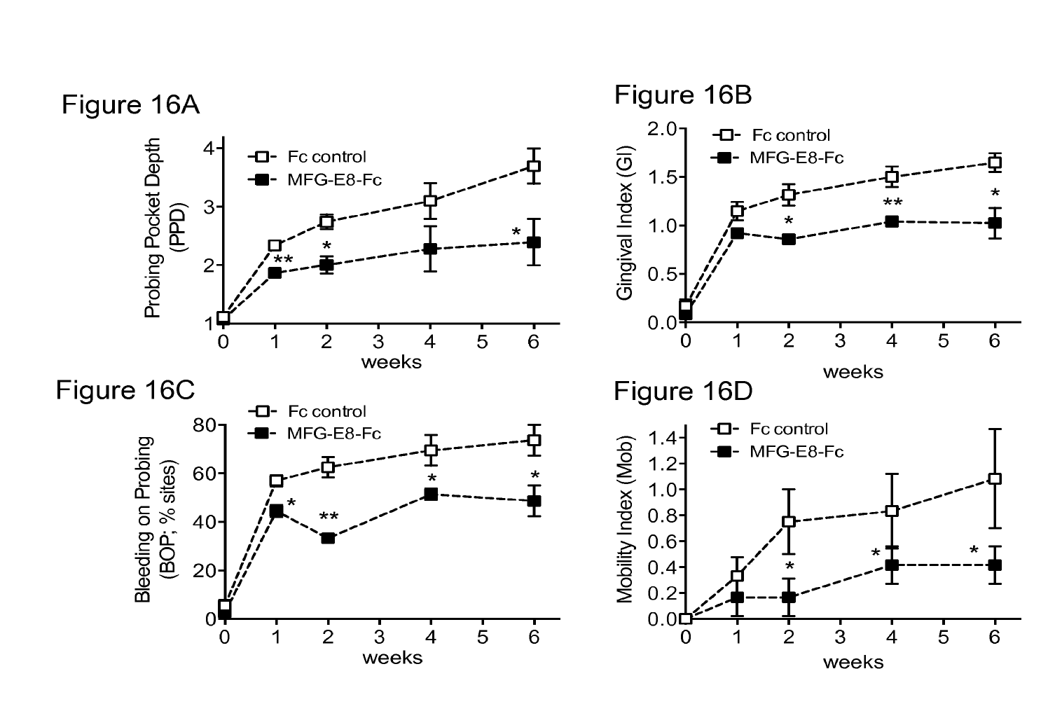

Starting three days after initiation of ligature-induced periodontitis, MFG-E8-Fc or Fc control were injected locally into the mandibular interdental papillae from the first premolar to the second molar, three times weekly, in opposites sides of the mouth (split-mouth design). The animals were clinically examined at the indicated timepoints and the effects of MFG-E8-Fc on the indicated inflammatory clinical parameters were recorded, probing pocket depth (FIG. 16A), gingival index (FIG. 16B), bleeding on probing (FIG. 16C) and mobility index (FIG. 16D). At the beginning of the study, the gingival margins in all animals were at the cement-enamel junction, and CAL readings equaled probing pocket depth (PPD). Data are means±SD (n=3 monkeys). *p<0.05; **p<0.01 compared with time-matched control (paired t test).

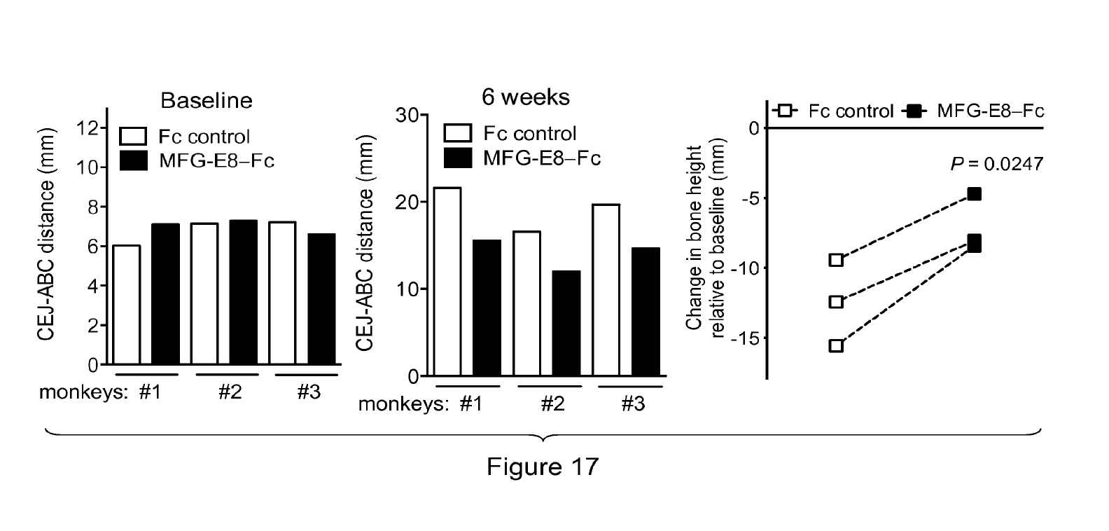

Three monkeys were treated as described in FIGS. 16A-16D, and their mandibular bone heights (CEJ-ABC distance) were measured using Nikon Imaging System software and standardized X-ray images (taken at baseline and at wk 6). Measurements were made at six points (first premolar, distal; second premolar, mesial and distal; first molar, mesial and distal; second molar, mesial) and the data in the left and middle graphs of FIG. 17 reflect the 6-site total at baseline and at wk 6, respectively. For each pair of Fc control and MFG-E8-Fc treatments, bone loss was calculated as bone height at baseline minus bone height at 6 wk (right graph of FIG. 17); the difference between control and Cp40 treatments was significant (p<0.05) (paired t test).

### MFG-E8 as a Biomarker for Human Periodontal Disease

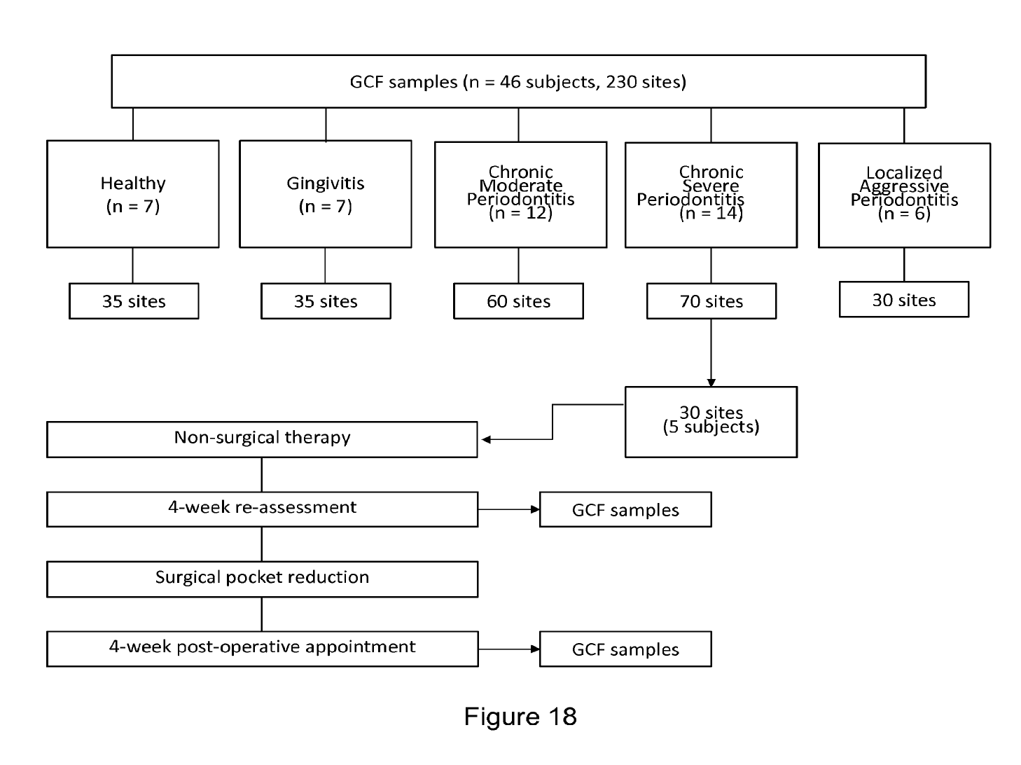

MFG-E8 and inflammatory cytokines were analyzed in human GCF from subjects classified as periodontally healthy, gingivitis, moderate and severe periodontitis as well as localized aggressive periodontitis. FIG. 18 shows the distribution of sites among all groups. 45.65% of the subjects were male and 54.35% were female with a mean age of 53.23 and a standard deviation of 4.33. The distribution of age and gender among groups can be found in Table 1. Additionally, Table 1 shows the probing pocket depth (PPD) measurements in all groups at time of initial examination. Table 2 shows the levels of the analytes which were detected in this study. These include MFG-E8, OPG, RANKL, IL-1a, IL-1β, IL-6 and IL-17A. Table 3 shows the reduction in (PPD) in the treated severe periodontitis subgroup, while Table 4 includes the cytokine levels of the analyses were detected in the treated severe periodontitis subgroup (MFG-E8, RANKL, IL-6 and IL-17A).

TABLE 1
Mean probing pocket depth (PPD), age and gender distribution
among groups at time of initial GCF collection.

| | Moderate | | Severe | | Aggressive | | | | | |
|---|---|---|---|---|---|---|---|---|---|---|
| | Periodontitis | | Periodontitis | | Periodontitis | | Gingivitis | | Healthy | |
| | Mean | SD | Mean | SD | Mean | SD | Mean | SD | Mean | SD |
| PPD | 4.13 | 1.24 | 5.60 | 1.55 | 6.70 | 1.58 | 2.57 | 0.78 | 2.09 | 0.56 |
| Age | 53.32 | 6.28 | 52.35 | 5.66 | 19.32 | 3.33 | 56.78 | 4.23 | 49.88 | 6.57 |
| Gender | Male | Female | Male | Female | Male | Female | Male | Female | Male | Female |
| (%) | 58.3% | 41.7% | 42.9% | 57.1% | 33.3% | 66.7% | 57.1% | 42.9% | 28.6% | 71.4% |

TABLE 2
Mean level of MFG-E8, OPG, RANKL, IL-1α, IL-1β, IL-6 and
IL-17A among groups at time of initial GCF collection.

| | Moderate | | Severe | | Aggressive | | | | | |
|---|---|---|---|---|---|---|---|---|---|---|
| | Periodontitis | | Periodontitis | | Periodontitis | | Gingivitis | | Healthy | |
| | Mean | SD | Mean | SD | Mean | SD | Mean | SD | Mean | SD |
| MFG-E8 | 27.00 | 5.26 | 12.38 | 5.88 | 10.60 | 5.32 | 43.82 | 5.67 | 41.76 | 6.77 |
| OPG | 120.93 | 37.50 | 93.69 | 29.72 | 75.65 | 36.69 | 175.68 | 53.84 | 162.10 | 55.36 |
| RANKL | 134.95 | 23.09 | 143.73 | 35.58 | 140.07 | 32.68 | 48.32 | 20.94 | 37.60 | 13.06 |
| IL-1α | 476.01 | 324.55 | 525.16 | 342.00 | 503.67 | 266.34 | 275.45 | 188.81 | 191.13 | 142.58 |
| IL-1β | 531.31 | 134.73 | 597.81 | 133.52 | 651.68 | 179.22 | 189.11 | 68.43 | 132.43 | 63.31 |
| IL-6 | 587.36 | 150.24 | 754.01 | 146.52 | 865.53 | 170.66 | 102.74 | 36.44 | 45.45 | 18.07 |
| IL-17A | 15.93 | 6.22 | 24.11 | 4.85 | 34.20 | 6.22 | 0.03 | 0.03 | 0.03 | 0.03 |

TABLE 3
Mean probing pocket depth (PPD) in severe periodontitis treatment
subgroup at time of initial GCF collection, 4-week re-evaluation
appointment after non-surgical therapy and 4-week postoperative
appointment after pocket reduction surgery.

| | | | |
|---|---|---|---|
| Treatment | Initial | Reevaluation | Post Surgery |

| Subgroup | Mean | SD | Mean | SD | Mean | SD |
|---|---|---|---|---|---|---|
| PPD | 6.03 | 1.61 | 3.97 | 1.07 | 2.63 | 0.67 |

TABLE 4
Mean levels of MFG-E8, RANKL, IL-6, IL-17A in severe periodontitis
treatment subgroup at time of initial GCF collection, 4-week
re-evaluation appointment after non-surgical therapy and 4-week
postoperative appointment after pocket reduction surgery.

| Treatment | Initial | | Reevaluation | | Post Surgery | |
|---|---|---|---|---|---|---|
| Subgroup | Mean | SD | Mean | SD | Mean | SD |
| MFG-E8 | 11.31 | 3.42 | 17.03 | 5.47 | 27.78 | 7.51 |
| RANKL | 149.29 | 30.27 | 107.92 | 23.61 | 67.67 | 16.41 |
| IL-6 | 799.76 | 138.28 | 323.20 | 178.82 | 152.70 | 69.42 |
| IL-17A | 25.64 | 5.15 | 21.64 | 5.55 | 12.16 | 4.93 |

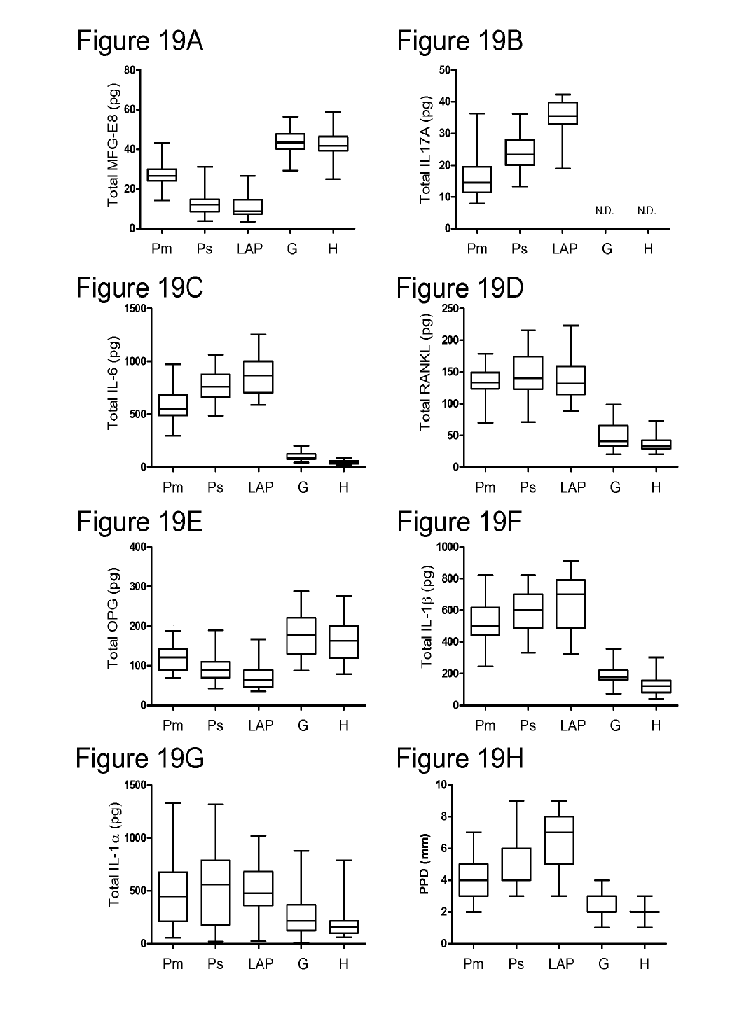

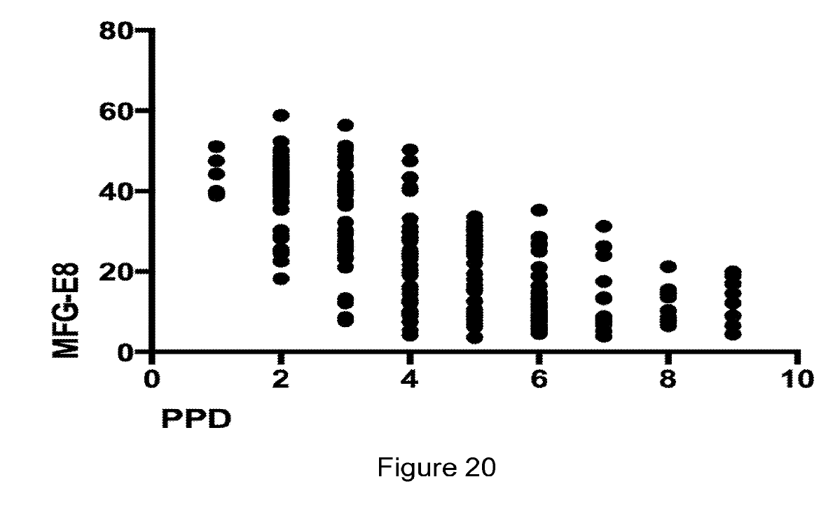

FIGS. 19A-H show the levels of detected analytes in all subject groups, as well as differences in PPD. No significant difference was found in the levels of MFG-E8 in gingivitis and in health (p>0.9999) but the levels in both groups were significantly higher than all periodontitis groups (p<0.0001). The moderate periodontitis group showed significantly higher levels of MFG-E8 than both the severe periodontitis group and the aggressive periodontitis group (p<0.0005), but no significant difference was found between levels in severe periodontitis and sites with localized aggressive periodontitis (p>0.9999). FIG. 20 shows the scatter plot of the correlation between PPD and MFG-E8 in all sites at initial examination. Spearman's rank correlation showed significant negative correlation between PPD and GCF MFG-E8 levels (r=−0.7101, p<0.0001).

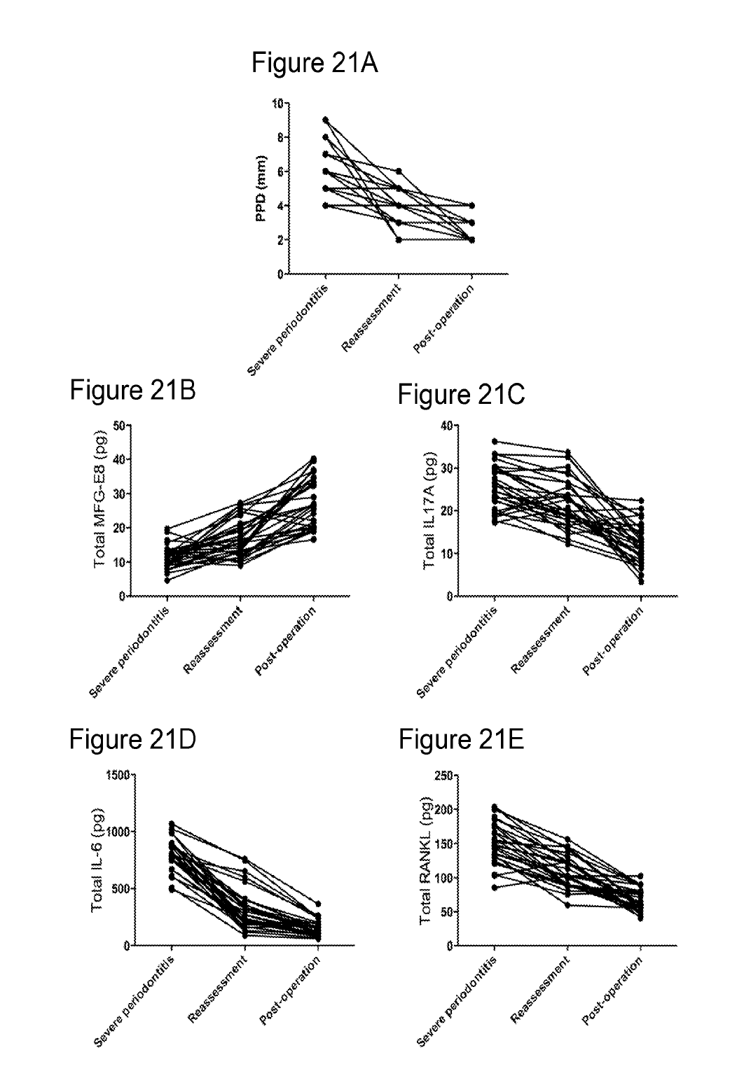

When MFG-E8 was examined after non-surgical and surgical therapy in the severe periodontitis group, it was found that the level of MFG-E8 in GCF increases with decreasing probing pocket depths. The level of MFG-E8 was significantly higher after non-surgical therapy and further increased significantly after surgical therapy as periodontal pockets were eliminated (p<0.0001) as seen in FIGS. 21A-21E.

The level of OPG was found to follow a trend similar to MFG-E8 levels. It was found to be significantly higher in health and gingivitis as compared to periodontitis groups (p<0.05). Moderate periodontitis had significantly higher levels of OPG when compared to severe (P=0.0026) and aggressive periodontitis (p<0.0001). No significant differences were found between OPG levels in severe and aggressive periodontitis (p=0.5604). RANKL, on the other hand, showed an opposing trend with significantly higher levels in the periodontitis groups as compared to health and gingivitis (P<0.0001). No significant differences were found between the health and gingivitis group (p>0.9999) or between all three periodontitis groups (p>0.9999). The level of RANKL was found to be reduced as probing depths were reduced. It was found to significantly reduce following non-surgical and surgical therapy (p<0.0001).

The level of IL-1a was also found to be significantly reduced in health and gingivitis as compared with periodontitis groups (p<0.0450) with no significant differences among all three periodontitis groups and no significant difference between health and gingivitis (p>0.9999). IL-1β was significantly lower in health and gingivitis groups as compared to the periodontitis groups (p<0.0001). No significant difference was found between health and gingivitis group (p>0.9999) or among the periodontitis groups (p<0.167).

IL-6 was barely detectable in health and gingivitis groups with no significant differences between both groups but was significantly increased in periodontitis groups (p<0.0001). The moderate periodontitis group had significantly lower levels of IL-6 as compared to the severe periodontitis group (p=0.0007) and the localized aggressive periodontitis group (p<0.0001). No significant difference was found between the severe and localized aggressive periodontitis groups (p=0.8767). IL-6 was also significantly reduced after surgical and non-surgical therapy (p<0.0001).

IL-17A was not detected in health and gingivitis but it was detected in all periodontitis groups with the highest level found in the localized aggressive periodontitis group. The level of IL-17A in the aggressive periodontitis groups was significantly higher than all other test groups (p<0.0001 for healthy, gingivitis and moderate periodontitis. P=0.0194 for severe periodontitis). Its level in the severe periodontitis group was significantly higher than the moderate periodontitis group (p=0.0003) and both the health and gingivitis groups (p<0.0001). Furthermore, the level of IL-17A in the moderate periodontitis group was significantly higher than the level in health and gingivitis (p<0.0001). IL-17A was found to be significantly reduced following non-surgical and surgical therapy (p<0.0001).

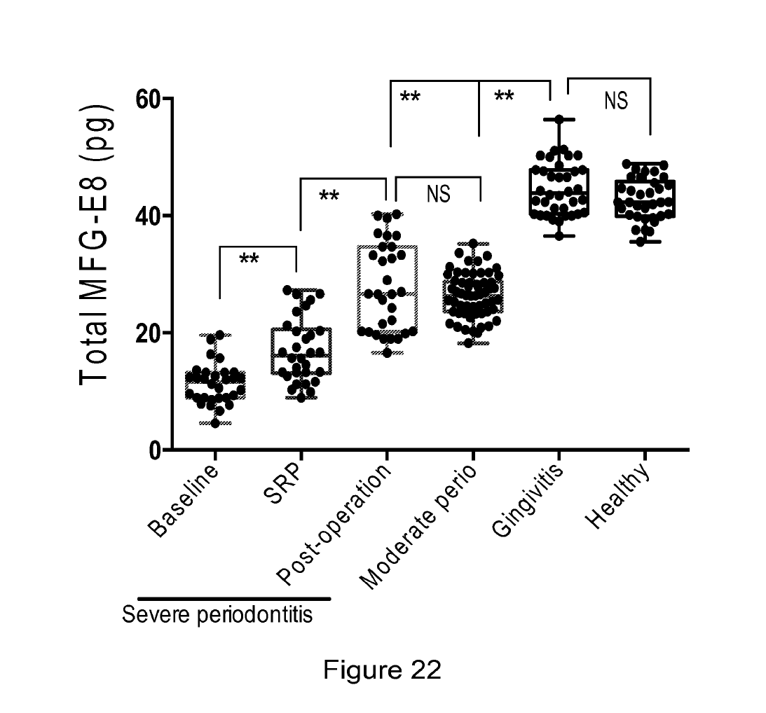

The results of the human study have shown that the secreted glycoprotein MFG-E8 can be detected in human gingival crevicular fluid in different states of health and disease using magnetic bead-based multiplexing assays. It was also found that the levels of MFG-E8 in human GCF were higher in cases of health and gingivitis than in periodontal disease, where they are inversely related to the severity of the disease, see FIG. 22. It was also shown to be decreased in areas affected with localized aggressive periodontitis.

Overall, the investigation of different cytokines, which play a role in periodontal disease, showed that the levels of pro-inflammatory and pro-osteoclastogenic cytokines IL-1α, IL-1β, IL-17A, RANKL were up regulated with increasing periodontal disease severity while the levels of homeostatic molecules, OPG and MFG-E8, were down regulated.

The results described herein have shown the presence of MFG-E8 in human GCF collected from healthy, gingivitis and periodontitis subjects by using a magnetic bead-based immunoassay.

The levels of MFG-E8 were negatively related to the level of gingival inflammation and were found to increase after surgical and non-surgical treatment of periodontal disease. These data support the value of MFG-E8 as a biomarker for periodontal inflammation.

The Materials and Methods used in the performance of the experiments disclosed herein are now described.

Mice and periodontitis model. Mfge8−/− mice were generated as previously described and were speed backcrossed to the C57BL/6NCr genotype to generate mice that were ≈99% identical to C57BL/6NCr mice. Colonies of Mfge8−/− and C57BL/6NCr WT controls (Charles River Laboratories) were established at the University of Pennsylvania. Mice were housed in a pathogen-free environment and used when they were 8-10 wk-old except in experiments of aging lasting up to the age of 13 mo. Ligature-induced periodontitis was performed and is described in detail herein. In intervention experiments, anti-MFG-E8 mAb (5 μg; clone B1F10) or rMFG-E8 (2.5 μg; R&D Systems) or equal amounts of corresponding controls (IgG2a and BSA, respectively) were microinjected into the palatal gingiva of the ligated second maxillary molar.

Osteoclastogenesis and Resorption Pit Formation.

RANKL-induced osteoclastogenesis was performed according to standard protocols using BM-derived monocyte/macrophage precursor cells or RAW264.7 precursor cells. TRAP+MNCs were imaged using a Nikon Eclipse NiE automated upright fluorescent microscope with an attached digital camera and met the criteria of authentic OCLs, manifested by expression of OCL differentiation markers and bone-resorbing activity on calcium phosphate-coated wells. OCL resorption activity was determined using Osteo Assay Surface plates following the protocol of the manufacturer (Corning). Briefly, mouse OCPs from BM were plated at a density of 1×10⁵ cells/well in a 96-well plate coated with inorganic bone biomaterial (crystalline calcium phosphate). The cells were cultured in the presence of M-CSF (100 ng/ml) with or without RANKL (50 ng/ml) for 4 days. Similarly, human OCPs prepared as above (see Osteoclastogenesis) were plated at a density of 5×10⁴ cells/well in a 96-well plate coated with Ca₃(PO₄)₂ and cultured with 20 ng/ml M-CSF, with or without 40 ng/ml RANKL, for 5 days. At the end of the incubation period, both mouse and human OCLs were removed by 5-min treatment with 10% bleach and resorptive areas were visualized by light microscopy. The total resorbed area was measured using Photoshop CS6

Measurement of Mineral Density by Micro-Computed Tomography.

Tissue mineral density of mouse tibiae was measured by micro-computed tomography (μCT) using the μCT35 system (Scanco Medical AG) at the University of Pennsylvania Center for Musculoskeletal Disorders. A 1.2-mm-thick region located distal to the proximal growth plate was scanned at a 6-μm resolution and microstructural parameters were obtained through three-dimensional reconstruction and segmentation (using a Gaussian filter and a global threshold of 3892 Hounsfield units) in the manufacturer-provided software. The μCT35 system was calibrated by scanning hydroxyapatite phantoms of known mineral density. A standard curve was then used to convert the data into a mineral density value (mg HA/cm³).

Histological TRAP staining. Maxillae with intact surrounding tissue were fixed in 4% paraformaldehyde, decalcified in Immunocal solution (Decal Chemical) for 14 days, and embedded in OCT compound. TRAP staining was performed on coronal sections (6-μm thick) using the leukocyte acid phosphatase kit (Sigma-Aldrich). Slides were viewed using a Nikon Eclipse NiE microscope and TRAP+MNCs were considered to be OCLs.

Immunofluorescence Histochemistry.

Maxillary sections prepared as above were stained with antibodies to MFG-E8 (18A2-G10, MBL) or cathepsin K (polyclonal; Abcam) followed by secondary reagents (AlexaFluor594-conjugated goat anti-hamster IgG or AlexaFluor488-conjugated goat anti-rabbit IgG; Life Technologies). The specificity of staining was confirmed using appropriate isotype control or non-immune IgG. Images were captured using a Nikon Eclipse NiE automated fluorescent microscope.

Quantitative Real-Time PCR (qPCR).

Total RNA was extracted from excised gingival tissue or cultured cells using TRIzol (InVitrogen) and quantified by spectrometry at 260 and 280 nm. The RNA was reverse-transcribed using the High Capacity RNA-to-cDNA Kit (Life Technologies) and qPCR with cDNA was performed using the Applied Biosystems 7500 Fast Real-Time PCR System according to the manufacturer's protocol (Life Technologies). Data were analyzed using the comparative (ΔΔCt) method. TaqMan probes, sense primers, and antisense primers for detection and quantification of genes investigated in this paper were purchased from Life Technologies.

Periodontitis Models.

(a) Ligature-Induced Periodontitis.

The placement of ligatures accelerates bacteria-mediated inflammation and bone loss (1). To induce bone loss, a 5-0 silk ligature was tied around the maxillary left second molar, as previously described (2). The contralateral molar tooth in each mouse was left unligated to serve as baseline control for bone loss measurements. The ligatures remained in place in all mice throughout the experimental period. The mice were euthanized at various timepoints (0 to 10 d) after placement of the ligatures and defleshed maxillae were used to measure bone heights (i.e., the distances from the cementoenamel junction (CEJ) to the alveolar bone crest (ABC) under a Nikon SMZ800 microscope using a 40 x objective. Images of the maxillae were captured using a Nicon Digital Sight DS-U3 camera controller and CEJABC distances were measured at 6 predetermined points on the ligated molar and adjacent regions using NIS-Elements software (Nikon Instruments Inc.) (2). To calculate bone loss, the 6-site total CEJ-ABC distance for the ligated side of each mouse was subtracted from the 6-site total CEJ-ABC distance of the contralateral unligated side. The results were presented in mm and negative values indicated bone loss relative to the baseline (unligated control).

(b) Naturally-Occurring Periodontitis.

Mfge8−/− mice and WT controls were reared in parallel from the age of 10 wk until 13 mo. Defleshed maxillae from euthanized mice were used for bone measurements. CEJ-ABC distances were measured on 14 predetermined points on maxillary molars (3). To calculate bone loss, the 14-site total CEJ-ABC distance for each mouse was subtracted from the mean CEJ-ABC distance of sham-infected mice.

Osteoclastogenesis.

(a) BM-Derived Precursors.

BM cells were flushed from femurs and tibias of mice. After lysis of erythrocytes using RBC lysis buffer (eBioscience), BM cells were cultured on petri dishes with recombinant murine M-CSF (5 ng/ml; R&D Systems) for 16 h. The nonadherent cell population was recovered and further cultured in α-MEM media/10% FBS with 100 ng/ml M-CSF for 3 d. Floating cells were removed and attached cells were used as BM-derived monocyte/macrophage precursor cells (“osteoclast precursors”; OCPs). OCPs (1×10⁵ per well in a 96-well plate) were cultured for 3 d in the presence of 50 ng/ml soluble recombinant RANKL (R&D Systems) and 100 ng/ml M-CSF to generate osteoclasts (OCLs). The cells were fixed and stained for TRAP using an acid phosphatase leukocyte diagnostic kit (Sigma-Aldrich) and TRAP+ multinucleated (≧3 nuclei) cells were counted (4). A Nikon Eclipse NiE automated upright fluorescent microscope with an attached digital camera was used to image the cells.

(b) RAW264.7 Cells.

To induce osteoclastogenesis from RAW264.7 cells, the cells were plated at a density of 2×10³ cells per well into a 96-well plate and cultured with α-MEM media/10^{%}FBS in the presence of 20 ng/ml RANKL for 4 d (M-CSF was not added as this cytokine is produced by RAW264.7 cells). Cultures were re-fed and re-treated with RANKL at d3 and TRAP+ multinucleated cells were counted the following day (5).

(c) Human Monocytes.

To generate human OCLs (6), CD14+ monocytes were isolated from human peripheral blood mononuclear cells (obtained from the University of Pennsylvania Human Immunology Core) using anti-CD14 magnetic beads as instructed by the manufacturer (StemCell Technologies). After incubation with M-CSF (20 ng/ml) for 24 h, the generated OCPs were added to 96-well plates at a seeding density of 5×10⁴ cells per well and incubated in α-MEM media/10^{%}FBS supplemented with M-CSF (20 ng/ml) and RANKL (40 ng/ml) for 5 d. Media and cytokines were replenished on d3. TRAP+ multinucleated cells were counted on d5. In experiments of mouse or human osteoclastogenesis designed to determine the effects of MFG-E8, MFG-E8 was added together with RANKL.

Determination of Bacterial Counts.

In ligature-induced periodontitis, the ligatures were recovered from euthanized mice and gently washed with PBS to remove food residue and other debris. Subsequently, the sutures were placed in Eppendorf tubes with 1 ml PBS and the bacteria were extracted by vortexing for 2 min at 3000 rpm. Serial dilutions of the bacterial suspensions were plated onto blood agar plates and CFU were enumerated following anaerobic growth at 37° C. for 7 d. Results were normalized by dividing CFU by the length (mm) of the corresponding suture. To assess the oral microbial burden in naturally-occurring periodontitis, the murine oral cavity was sampled for 1 min using sterile swabs held against the gumlines and the extracts were processed as above for CFU enumeration.

Antimicrobial Activity.

The disk inhibition zone assay was used to determine possible antimicrobial activity of MFG-E8 using Imipenem and PBS as positive and negative control, respectively. The assay was performed according to the Performance Standards for Antimicrobial Susceptibility Testing (Twenty First Informational Supplement, M100S21). A total of 15 anaerobic bacterial isolates were randomly selected from ligature-induced periodontal lesions of 5 mice (3 isolates per mouse). Sterile filter paper discs (7-mm diameter; 185-am thickness) were impregnated with various amounts of the test and control compounds and placed on Gifu anaerobic medium (GAM)-based blood agar plates (Nissui Pharmaceutical), which had been previously spread with 100 al of inocula, each containing bacterial suspension equivalent to 0.5 McFarland standard. The plates were incubated at 37° C. for 7 d and the diameter of the growth inhibition zones around the discs was measured using a vernier caliper.

Immunoprecipitation and Immunoblotting.

Cell lysates were prepared using the RIPA Lysis Buffer System (Santa Cruz Biotechnology) and protein content concentrations were determined by Nano Drop 2000C spectrophotomer (Thermo Scientific). Immunoprecipitation was carried out using goat polyclonal anti-MFG-E8 antibody or non-immune IgG control (R&D Systems) and protein G-coupled magnetic beads according to the manufacture's protocol (Life Technologies). Proteins were separated by standard SDS-PAGE on 10% acrylamide gels (Life Technologies) and transferred to polyvinylidene difluoride membrane (Bio-Rad) by electroblotting. The membranes were incubated in blocking buffer (5% nonfat dried milk, 10 mM Tris [pH 7.5], 100 mM NaCl, and 0.05% Tween 20) followed by probing with goat polyclonal anti-MFG-E8 antibody (R&D Systems) or anti-MFG-E8 mAb (18A2-G10; MBL) and visualization with horseradish peroxidase-conjugated secondary antibody and chemiluminescence using the Amersham Biosciences ECL system. Images were captured using a FluorChem M imaging system (ProteinSimple).

Statistical Analysis.

Data were evaluated by ANOVA and the Dunnett multiple-comparison test using the InStat program (GraphPad). Where appropriate (comparison of two groups only), twotailed t tests were performed. All experiments were performed two or more times for verification.

Study Approval.

All animal procedures were performed according to protocols approved by the Institutional Animal Care and Use Committee of the University of Pennsylvania.

siRNA Silencing.

Short-interfering RNAs (siRNAs) against MFG-E8 and Del-1 were synthesized by Ambion (Life Technologies, Carlsbad, Calif.). For transient transfections of siRNA, each pair of oligoribonucleotides was annealed at a concentration of 10 nM and transfected into cells in 6-well plates using Lipofectamine RNAiMAX reagent (Life Technologies, Carlsbad, Calif.), according to the manufacturer's protocol. The Ambion's silencer negative control siRNA was used to confirm that the transfection did not cause nonspecific effects on gene expression.

Participants.

A group of patients presenting to the Penn Dental Medicine Graduate Periodontal Clinic, The University of Pennsylvania, Philadelphia, Pa. were recruited to participate in this cross-sectional study from January 2013 to June 2014. The subjects enrolled were grouped as healthy (H), gingivitis (G), moderate periodontitis (Pm), severe periodontitis (Ps) or localized aggressive periodontitis (LAP).

The main inclusion criteria were adult subjects with good general health and with at least 10 teeth in the functional dentition excluding third molars and who had never received periodontal treatment at the time of initial clinical examination. All subjects were required to be able to read and understand the written consent form. For patients with periodontitis, at least two quadrants had to involve deep probing depths (PD), clinical attachment loss (CAL) and detectable radiographic bone loss.

Subjects with the following conditions were excluded:
1. Smoking, drug or alcohol abuse.
2. Uncontrolled diabetes.
3. Previous head and neck radiotherapy.
4. History of chemotherapy in the previous 12 months.
5. Immunocompromised subjects or subjects suffering from systemic diseases that significantly affect the periodontium.
6. Subjects taking medications known to affect the periodontium (e.g. Phenytoin, calcium channel blockers, cyclosporine . . . etc).
7. Pregnant or lactating females.
8. Subjects requiring prophylactic antibiotics.
9. Subjects taking steroid medications except for acute topical treatment.
10. Subjects using systemic antibiotics within three months prior to enrollment.
11. Subjects who currently have, or history of (within three months), of the following diseases: severe cardiovascular, pulmonary or liver diseases, end stage renal disease, active malignancy, cerebral vascular disease, HIV, TB, hepatitis or other active infectious diseases.

All subjects who volunteered to participate underwent routine periodontal examination and were classified according to the Armitage Classification System as either having plaque-induced gingivitis, chronic moderate periodontitis, chronic severe periodontitis, localized aggressive periodontitis or being periodontally healthy.

Subjects with PD<3 mm, no signs of gingival inflammation, CAL, bleeding on probing (BOP) or radiographic bone loss were placed in the healthy group. Subjects with PD<3 mm, with signs of gingival inflammation or BOP but without CAL or radiographic bone loss were placed in the gingivitis group. The Moderate Periodontitis group included subjects with CAL of 3-4 mm and moderate radiographic bone loss. Subjects with CAL of 5 mm or more with severe radiographic bone loss were placed in the severe periodontitis group. Subjects with minimal amount of gingival inflammation and BOP with CAL and radiographic bone loss that was limited to the first molars and incisors were placed in the aggressive periodontitis group.

This study was approved by the IRB of the University of Pennsylvania (IRB #817153). The protocol was explained to the subjects and verbal and written consent was taken in accordance with the Declaration of Helsinki.

Sample Collection.

All samples were collected after reviewing the medical history and performing clinical and radiographic periodontal examination and establishing a periodontal diagnosis. PD and CAL were measured at 6 sites for every tooth using a manual probe (UNC 15, Hu-Friedy, Chicago, Ill., USA). Five teeth were selected from each subject with at least one anterior tooth and two posterior teeth from two different quadrants. The teeth were dried and isolated with cotton rolls and the samples were obtained from the surface with the deepest PD. Samples were collected using absorbent paper strips (Periopaper, ProFlow Inc., Amityville, N.Y., USA) which were placed in the selected sites until mild resistance was felt and kept in place for 30 seconds. Samples with blood or saliva contamination were discarded. The individual paper strips were subsequently transferred into polypropelene tubes (Eppendorf, Hamburg, Germany) and were frozen (−80° C.) until they were ready to be analyzed.

GCF Samples.

A total of 46 subjects were included in the analysis with a total of 230 sites. The site distribution was as follows: 35 sites (7 subjects) in the Healthy group, 35 sites (7 subjects) in the Gingivitis group, 60 sites (12 subjects) in the moderate periodontitis group, 70 sites (14 subjects) in the severe periodontitis group and 30 sites (6 subjects) in the aggressive periodontitis group. Thirty samples (5 subjects) from the severe periodontitis group were reexamined at reevaluation appointment after non-surgical periodontal treatment consisting of scaling and root planing and home care oral hygiene instructions as well as after pocket reduction surgery (FIG. 1). At the time of analysis, the contents of the GCF samples were eluted from the paper strips using phosphate buffered solution. The level of MFG-E8 and other cytokines were analyzed using Luminex® xMAP® multiplexing bead-based immunoassays utilizing magnetic carboxylated polystyrene microspheres (MagPlex™).

MFG-E8. MFG-E8 was quantified with a Human Premixed Multi-Analyte Kit (R&D Systems, Minneapolis, Minn., USA). The assay was done according to manufacturer's instructions. The diluted magnetic micro particle cocktail was added to the wells of a 96-well magnetic plate. Samples and standards were added in duplicate to the magnetic beads and incubated for 2 hours at room temperature with shaking on a plate shaker. The wells were then washed three times with the supplied Wash Buffer using a magnetic plate holder. Diluted Biotin Antibody Cocktail was then added to each well and the plate was incubated at room temperature on a plate shaker for 1 hour. After that, the wash procedure was repeated and diluted Streptavidin-PE was added to each well. The plate was sealed and incubated for 1 hour on a shaker at room temperature. The plate was then washed and the microparticles were resuspended in Wash Buffer and incubated on a shaker for 2 minutes. The plate was then ready to be read by the Milliplex Analyzer (EMD Millipore, Darmstadt, Germany).

Other Analytes.

All samples were also analyzed using HCYTMAG-60K-PX30 Human Cytokine/Chemokine Magnetic Bead Panel Kit (EMD Millipore, Darmstadt, Germany) to detect levels of Eotaxin, G-CSF, GM-CSF, IFN-γ, IL-1a, IL-1β, IL-1ra, IL-2, IL-3, IL-4, IL-5, IL-6, IL-7, IL-8, IL-10, IL-12 (p40), IL-12 (p70), IL-17, TNF-α and TNF-β. The assay was performed according to the manufacturer's instructions. The diluted Wash Buffer was added to the wells of a 96-well plate and the plate was sealed and shaken on a plate shaker for 10 minutes at room temperature. The plate was then decanted and dried. Standards and Controls were added in duplicate to the specified wells followed by PBS. Diluted Assay Buffer was added to the sample wells followed by the samples which were also added in duplicate. After that, the premixed beads were added to each well and the plate was sealed and incubated on a plate shaker overnight at 4° ° C. At the following day, the plate contents were removed using a magnetic plate holder to retain the magnetic beads and the plate was washed twice using the Wash Buffer. Detection antibodies were placed into each well and the plate was sealed and incubated with agitation on a plate shaker for 1 hour at room temperature. After incubation, Streptavidin-Phycoerythrin was added to each well and the plate was incubated with agitation for 30 minutes at room temperature. The washing procedure was repeated twice. The beads were resuspended by the addition of Sheath Fluid and agitation for 5 minutes and the plate was run by the Milliplex Analyzer (EMD Millipore, Darmstadt, Germany).

The samples were also processed a third time using Human Bone Magnetic Bead Panel (EMD Millipore, Darmstadt, Germany) to detect RANKL, OPG and OPN following the same procedure as the human cytokine/chemokine kit.

Statistical Analysis.

Data were analyzed using Graphpad Prism software. Comparison between groups was done using Kruskal-Wallis test with Dunn's multiple comparison test for each detected analyte. Correlation between MFG-E8 and PPD was done using Spearman's rank-order correlation. In the treatment subgroup, differences after nonsurgical treatment were checked using Wilcoxon signed ranks test that was repeated for each analyte. p-value of 5% was considered significant.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising an effective amount of milk fat globule-EGF factor 8 (MFG-E8) formulated for local administration and a pharmaceutically acceptable carrier or adjuvant.

2. The composition of claim 1, wherein the composition is formulated for oral administration and is selected from the group consisting of a liquid suspension, a chewable composition, and an orally disintegrating tablet or capsule composition.

3. (canceled)

4. The composition of claim 1, wherein the composition is formulated for delayed-release.

5. The composition of claim 1, wherein the composition is formulated to target a site of bone loss selected from the group consisting of periodontal tissue, alveolar process, an arthritic joint, a non-arthritic joint, and an injured bone.

6. (canceled)

7. The composition of claim 1, wherein the effective amount is in a range of about 0.1 μg/ml to about 2 μg/ml per single dose.

8. The composition of claim 1 further comprising at least one binder, excipient, diluent, or any combination thereof.

9. The composition of claim 1 further comprising at least one of an antimicrobial agent and an anti-inflammatory agent.

10. A composition for treating a bone disorder comprising an effective amount of milk fat globule-EGF factor 8 (MFG-E8) formulated for local administration.

11. The composition of claim 10, wherein the effective amount inhibits at least one condition selected from the group consisting of osteoclastogenesis, inflammation and bone resorption.

12. A method of regulating osteoclast activation at a target site comprising locally administering an effective amount of milk fat globule-EGF factor 8 (MFG-E8) to the target site.

13. A method of inhibiting bone loss at a target site comprising locally administering an effective amount of milk fat globule-EGF factor 8 (MFG-E8) to the target site.

14. The method of claim 12 or claim 13, wherein the administration inhibits expression of at least one osteoclast marker selected from the group consisting of NFATc1, cathepsin K, and αvβ3 integrin.

15. (canceled)

16. The method of claim 12 or claim 13, wherein the administration inhibits osteoclastogenesis comprising RANKL-induced osteoclastogenesis.

17. (canceled)

18. The method of claim 12 or claim 13, wherein the administration inhibits bone resorption comprising at least one bone resorption stimulator selected from the group consisting of TNF, IL-6, IL-17A, MMP-9, Ptgs2, RANKL, IL-17A, Tnfsf11, CXCL1, CXCL2, CXCL3, CXCL5, and combinations thereof.

19. (canceled)

20. (canceled)

21. The method of claim 12 or claim 13, wherein the administration inhibits expression of at least one proinflammatory cytokine selected from the group consisting of IL-8 and CCL2/MCP-1.

22. The method of claim 12 or claim 13, wherein the target site is selected from the group consisting of periodontal tissue, alveolar process, an arthritic joint, a non-arthritic joint, and an injured bone.

23. A method of inhibiting inflammation at a target site comprising locally administering an effective amount of milk fat globule-EGF factor 8 (MFG-E8) to the target site.

24. The method of claim 23, wherein the inhibition decreases expression of at least one inflammation molecule selected from the group consisting of a pro-inflammatory mediator, an adhesion molecule, an immune receptor, IL-6, IL-8, IL-17a, MMP9, PTGS2, TNFSF11, SPP1, CSF3, CXCL1, CXCL2, CXCL3, CXCL5, ITGAL, SELE, CXCR2, CCR1, and TREM1.

25. (canceled)

26. The method of claim 23, wherein the target site is selected from the group consisting of periodontal tissue, alveolar process, an arthritic joint, a non-arthritic joint, and an injured bone.

27. The method of claim 26, wherein the target site is the periodontal tissue and the administration is in a gingival tissue.

28. The method of claim 27, wherein the inhibition suppresses periodontal microbiota growth.

29. A method of treating a bone disorder comprising locally administering an effective amount of milk fat globule-EGF factor 8 (MFG-E8) to a target site.

30. The method of claim 29, wherein the bone disorder is selected from the group consisting of osteoporosis, osteomalacia, osteosclerosis, and osteopetrosis.

31. Use of a composition comprising an effective amount of milk fat globule-EGF factor 8 (MFG-E8) formulated for local administration and a pharmaceutically acceptable carrier or adjuvant for treating a bone disorder.
